(19) United States
(12) Patent Application Publication
(10) Pub. No.: US 2009/0297596 A1
(43) Pub. Date: Dec. 3, 2009

(54) Nanoparticulate and Controlled Release Compositions Comprising a Platelet Aggregation Inhibitor

(75) Inventors: John Devane, Athlone (IE); Paul Stark, Glasson (IE); Niall Fanning, Dublin (IE); Gurvinder Rekhi, Suwanee, GA (US); Scott Jenkins, Downingtown, PA (US); Gary Liversidge, West Chester, PA (US)

Correspondence Address: Fox Rothschild, LLP;Elan Pharma International Limited, 2000 Market Street, Philadelphia, PA, 19103, US

(73) Assignee: ELAN PHARMA INTERNATIONAL LIMITED, Athlone (IE)

(21) Appl. No.: 11/568,835

(22) PCT Filed: May 23, 2006
(86) PCT No.: PCT/US06/19905

§ 371 Date: Apr. 6, 2009

Related U.S. Application Data
(60) Provisional application No. 60/683,620, filed on May 23, 2005.

(51) Int. Cl. A61K 9/64; A61K 9/20; A61K 9/10; A61K 9/14; A61K 31/47

(52) U.S. Cl. 424/456; 424/464; 424/484; 424/489; 424/490; 424/501; 514/312; 977/906

(57) ABSTRACT

The present invention provides a composition comprising a platelet aggregation inhibitor, for example, cilostazol, or a salt or derivative thereof, useful in the treatment and prevention of ischemic symptoms. The invention provides a composition which comprises nanoparticulate particles comprising the platelet aggregation inhibitor and at least one surface stabilizer. The nanoparticulate particles have an effective average particle size of less than about 2000 nm. The invention provides also a composition that delivers a platelet aggregation inhibitor, or nanoparticles comprising the same, in a pulsatile or continuous manner.

### FIELD OF INVENTION

The present invention relates to compositions and methods for the prevention and treatment of ischemic symptoms. In particular, the present invention relates to a composition comprising a platelet aggregation inhibitor and methods for making and using such a composition. In an embodiment of the invention, the composition is in nanoparticulate form and comprises also at least one surface stabilizer. The present invention relates also to novel dosage forms for the controlled delivery of a platelet aggregation inhibitor. Platelet aggregation inhibitors for use in the present invention include cilostazol, and salts and derivatives thereof.

### BACKGROUND OF INVENTION

Intermittent claudication is pain in the legs that occurs with walking and disappears with rest. It occurs because narrowing or blockage of the arteries decreases blood flow to the legs resulting in insufficient levels of oxygen in leg muscles during exercise.

Platelet aggregation inhibitors reduce the pain of ischemic symptoms by dilating the arteries, thereby improving the flow of blood and oxygen. Specifically, in the case of intermittent claudication, platelet aggregation inhibitors improve the flow, of blood and oxygen to the legs and enable patients to walk longer and faster before developing pain.

Cilostazol is an anti-platelet agent, a vasodilator, and a platelet aggregation inhibitor that has been shown to be effective for use in the prevention and treatment of ischemic symptoms such as intermittent claudication. Although its mechanism of action is not entirely clear, cilostazol inhibits phosphodiesterase III and suppresses cAMP degradation. These events result in increased levels of cAMP in platelets and blood vessels, leading to inhibition of platelet aggregation and vasodilation, respectively. In addition to its reported vasodilator and anti-platelet effects, cilostazol reduces the ability of blood to clot and has been proposed to have beneficial effects on plasma lipoproteins. By inhibiting the blood platelets from coagulating or aggregating, blood flow is enhanced and increased.

Cilostazol has been described in, for example, U.S. Pat. No. 4,277,479 for “Tetrazolylalkoxycarbostyril Derivatives and Pharmaceutical Compositions Containing Them”, U.S. Pat. No. 6,187,790 for “Use of Cilostazol for Treatment of Sexual Dysfunction”, U.S. Pat. No. 6,515,128 for “Processes for Preparing Cilostazol”, U.S. Pat. Nos. 6,531,603, 6,573,382, 6,531,603, 6,657,061, and 6,660,864 all for “Polymorphic Forms of 6-[4-1(1-Cyclohexyl-1H-tetrazol-5-yl)Butoxy]-3,4-Dihydro-2(1H)-Quinolinone”, U.S. Pat. Nos. 6,525,201, 6,660,773, and 6,740,758 all for “Processes for Preparing 6-Hydroxy-3,4-Dihydroquinolinone, Cilostazol and N-(4-Methoxyphenyl)-3-Chloropropionamide”, and U.S. Pat. No. 6,825,214 for “Substantially Pure Cilostazol and Processes for Making Same”.

The empirical formula of cilostazol is C₂₀H₂₇N₅O₂, and its molecular weight is 369.46. The chemical name of cilostazol is 6-[4-(1-cyclohexyl-1H-tetrazol-5-yl)butoxy]-3,4-dihydro-2(1H)-quinolinone, and it has the following chemical structure:

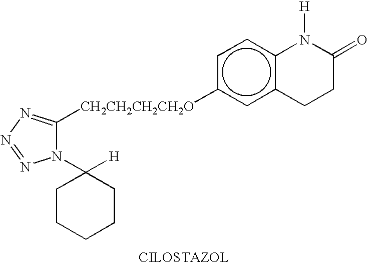

Cilostazol occurs as white to off-white crystals or as a crystalline powder that is freely soluble in chloroform, soluble in dimethylformamide, benzyl alcohol, and a mixture of chloroform and methanol (1:1), slightly soluble in methanol and ethanol, and is practically insoluble in water and absolute ether.

Cilostazol may be administered as part of a dosage form offered under the registered trademark name PLETAL® in the United States by Otsuka Pharmaceutical Co., Ltd. of Japan. PLETAL® tablets are available in 50 mg triangular and 100 mg round, white debossed tablets. The usual adult dosage of PLETAL® tablets is 100 mg twice daily, by the oral route. High fat meals increase the absorption of PLETAL®, and thus PLETAL® should be taken after a meal. PLETAL® is indicated for the reduction of symptoms of intermittent-claudication, as indicated by an increased walking distance.

Platelet aggregation inhibitors, such as cilostazol and the salts and derivatives thereof, have high therapeutic value for the treatment of patients suffering from ischemic symptoms. However, given the need to take such inhibitors two times a day and the further need to take such inhibitors after meals, strict patient compliance is a critical factor in the efficacy of such inhibitors in the treatment of ischemic symptoms. Moreover, such frequent administration often requires the attention of health care workers and contributes to the high cost associated with treatments involving platelet aggregation inhibitors. Thus, there is a need in the art for platelet aggregation inhibitor compositions which overcome the administration, compliance and other problems associated with their use in the treatment of ischemic symptoms.

The present invention fulfills such a need by providing a nanoparticulate composition comprising a platelet aggregation inhibitor, for example, cilostazol, or a salt or derivative thereof, and at least one surface stabilizer, which overcomes the poor bioavailability of platelet aggregation inhibitors and eliminates the requirement to take the platelet aggregation inhibitor with food. The present invention provides also a controlled release composition comprising a platelet aggregation inhibitor, for example, cilostazol, or a salt or derivative thereof, which eliminates the need to take the platelet aggregation inhibitor two times a day.

### SUMMARY OF THE INVENTION

The present invention relates to a nanoparticulate composition comprising: (A) a platelet aggregation inhibitor; and (B) at least one surface stabilizer. The composition may optionally comprise also a pharmaceutically acceptable carrier and any desired excipients. The surface stabilizer can be adsorbed on or associated with the surface of the nanoparticulate particles. The nanoparticulate particles have an effective average particle size of less than about 2,000 nm. A preferred dosage form of the invention is a solid dosage form, although any pharmaceutically acceptable dosage form can be utilized.

One embodiment of the invention encompasses a nanoparticulate composition comprising a platelet aggregation inhibitor wherein the pharmacokinetic profile of the platelet aggregation inhibitor, following administration of the composition to a subject, is not affected by the fed or fasted state of the subject.

In yet another embodiment, the invention encompasses a nanoparticulate composition comprising a platelet aggregation inhibitor wherein administration of the composition to a subject in a fasted state is bioequivalent to administration of the composition to a subject in a fed state.

Another embodiment of the invention is directed to a nanoparticulate composition comprising a platelet aggregation inhibitor and comprising also one or more additional compounds useful in the prevention and treatment of ischemic symptoms.

This invention further provides a method of making the inventive nanoparticulate composition. Such a method comprises contacting nanoparticulate particles comprising a platelet aggregation inhibitor with at least one surface stabilizer for a period of time and under conditions sufficient to provide a stabilized nanoparticulate composition comprising a platelet aggregation inhibitor.

The present invention is also directed to methods of treatment including but not limited to, the prevention and treatment of ischemic symptoms using the novel nanoparticulate compositions disclosed herein. Such methods comprise administering to a subject a therapeutically effective amount of such a composition. Other methods of treatment using the nanoparticulate compositions of the invention are known to those of skill in the art.

The present invention further relates to a controlled release composition comprising a platelet aggregation inhibitor which in operation produces a plasma profile substantially similar to the plasma profile produced by the administration of two or more IR dosage forms of a platelet aggregation inhibitor given sequentially. The platelet aggregation inhibitor may be contained in nanoparticulate particles which comprise also at least one surface stabilizer.

Conventional frequent dosage regimes in which an immediate release (IR) dosage form is administered at periodic intervals typically gives rise to a pulsatile plasma profile. In this case, a peak in the plasma platelet aggregation inhibitor concentration is observed after administration of each IR dose with troughs (regions of low platelet aggregation inhibitor concentration) developing between consecutive administration time points. Such dosage regimes (and their resultant pulsatile plasma profiles) have particular pharmacological and therapeutic effects associated with them. For example, the wash out period provided by the fall off of the plasma concentration of the active between peaks has been thought to be a contributing factor in reducing or preventing patient tolerance to various types of platelet aggregation inhibitors.

The present invention further relates to a controlled release composition comprising a platelet aggregation inhibitor which in operation produces a plasma profile that eliminates the “peaks” and “troughs” produced by the administration of two or more IR dosage forms given sequentially if such a profile is beneficial. This type of profile can be obtained using a controlled release mechanism that allows for continuous delivery. The platelet aggregation inhibitor may be contained in nanoparticulate particles which comprise also at least one surface stabilizer.

Multiparticulate modified controlled release compositions similar to those disclosed herein are disclosed and claimed in the U.S. Pat. Nos. 6,228,398 and 6,730,325 to Devane et al; both of which are incorporated by reference herein. All of the relevant prior art in this field may also be found therein.

It is a further object of the invention to provide a controlled release composition which in operation delivers a platelet aggregation inhibitor or nanoparticles containing the same, in a pulsatile manner or a continuous manner.

Another object of the invention is to provide a controlled release composition which substantially mimics the pharmacological and therapeutic effects produced by the administration of two or more IR dosage forms given sequentially.

Another object of the invention is to provide a controlled release composition which substantially reduces or eliminates the development of patient tolerance to a platelet aggregation inhibitor.

Another object of the invention is to provide a controlled release composition which releases an active ingredient therein in a bimodal manner. This may be accomplished, for example, in a composition in which a first portion of the active ingredient of the composition is released immediately upon administration and a second portion of the active ingredient is released rapidly after an initial delay period.

Another object of the invention is to formulate the dosage forms of a platelet aggregation inhibitor as an erodible formulation, a diffusion controlled formulation, or an osmotic controlled formulation.

Another object of the invention is to provide a controlled release composition capable of releasing a platelet aggregation inhibitor or nanoparticles containing the same, in a bimodal or multi-modal manner in which a first portion of the platelet aggregation inhibitor, or nanoparticles containing the same, is released either immediately or after a delay time to provide a pulse of platelet aggregation inhibitor release and one or more additional portions of the platelet aggregation inhibitor, or nanoparticles containing the same, is released, after a respective lag time, to provide additional pulses of the platelet aggregation inhibitor release during a period of up to twenty-four hours.

Another object of the invention is to provide solid oral dosage forms comprising a controlled release composition comprising a platelet aggregation inhibitor. The platelet aggregation inhibitor may be contained in nanoparticulate particles which comprise also at least one surface stabilizer.

Other objects of the invention include provision of a once daily dosage form of a platelet aggregation inhibitor which, in operation, produces a plasma profile substantially similar to the plasma profile produced by the administration of two immediate release dosage forms thereof given sequentially and a method for prevention and treatment of ischemic symptoms based on the administration of such a dosage form. The platelet aggregation inhibitor may be contained in nanoparticulate particles which comprise also at least one surface stabilizer.

The above objects are realized by a controlled release composition having a first component comprising a first population of a platelet aggregation inhibitor or nanoparticles containing the same, and a second component or formulation comprising a second population of a platelet aggregation inhibitor or nanoparticulates containing the same. The ingredient-containing particles of the second component further comprises a modified release constituent comprising a release coating or release matrix material, or both. Following oral delivery, the composition in operation delivers the platelet aggregation inhibitor or nanoparticulates containing the same, in a pulsatile or continuous manner.

The present invention utilizes controlled release delivery of a platelet aggregation inhibitor or nanoparticulates containing the same, from a solid oral dosage formulation, to allow dosage less frequently than before; and preferably once-a-day administration, increasing patient convenience and compliance. The mechanism of controlled release would preferably utilize, but not be limited to, erodible formulations, diffusion controlled formulations and osmotic controlled formulations. A portion of the total dose may be released immediately to allow for rapid onset of effect. The invention is useful in improving patient compliance and, therefore, therapeutic outcome for all treatments requiring a platelet aggregation inhibitor including but not limited to, the prevention and treatment of ischemic symptoms. This approach can replace conventional platelet aggregation inhibitor tablets and solutions, which are administered two times a day as adjunctive therapy in the prevention and treatment of ischemic symptoms.

The present invention also relates to a controlled modified release composition for the controlled release of a platelet aggregation inhibitor or nanoparticles containing the same. In particular, the present invention relates to a controlled release composition that in operation delivers a platelet aggregation inhibitor or nanoparticles containing the same, in a pulsatile or continuous manner, preferably during a period of up to twenty-four hours.

The present invention further relates to solid oral dosage forms containing a controlled release composition.

Preferred controlled release formulations are erodible formulations, diffusion controlled formulations and osmotic controlled formulations. According to the invention, a portion of the total dose may be released immediately to allow for rapid onset of effect, with the remaining portion of the total dose released over an extended time period. The invention is useful in improving compliance and, therefore, therapeutic outcome for all treatments requiring a platelet aggregation inhibitor including but not limited to, prevention and treatment of ischemic symptoms.

The invention relates further to nanoparticulate compositions of the type described above and controlled release compositions of the type described above in which the platelet aggregation inhibitor is cilostazol, or a salt or derivative thereof.

The present invention relates also to multiparticulate compositions of the type described above in which the nanoparticulate particles themselves form the particles of the multiparticulate.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

The present invention is described herein using several definitions, as set forth below and throughout the application.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, the phrase “therapeutically effective amount” shall mean the platelet aggregation inhibitor dosage that provides the specific pharmacological response for which the platelet aggregation inhibitor is administered in a significant number of subjects in need of the relevant treatment. It is emphasized that a therapeutically effective amount of a platelet aggregation inhibitor that is administered to a particular subject in a particular instance will not always be effective in treating the conditions/diseases described herein, even though such dosage is deemed to be a therapeutically effective amount by those of skill in the art.

The term “particulate” as used herein refers to a state of matter which is characterized by the presence of discrete particles, pellets, beads or granules irrespective of their size, shape or morphology.

The term “multiparticulate” as used herein means a plurality of discrete, or aggregated, particles, pellets, beads, granules or mixture thereof irrespective of their size, shape or morphology. A composition comprising a multiparticulate is described herein as a “multiparticulate composition”.

The term “nanoparticulate” refers to a multiparticulate in which the “effective average particle size” (see below) of the particles therein is less than about 2000 nm (2 microns) in diameter. A composition comprising a nanoparticulate is described herein as a “nanoparticulate composition”.

The phrase “effective average particle size” as used herein to describe a multiparticulate (e.g., a nanoparticulate) means that at least 50% of the particles therein are of a specified size. Accordingly, “effective average particle size of less than about 2000 nm in diameter” means that at least 50% of the particles therein are less than about 2000 nm in diameter.

“D50” refers to the particle size below which 50% of the particles in a multiparticulate fall. Similarly, “D90” is the particle size below which 90% of the particles in a multiparticulate fall.

The term “modified release” as used herein includes a release which is not immediate and includes controlled release, extended release, sustained release and delayed release.

The term “time delay” as used herein refers to the period of time between the administration of a dosage form comprising the composition of the invention and the release of the active ingredient from a particular component thereof.

The term “lag time” as used herein refers to the time between the release of the active ingredient from one component of the composition and the release of the active ingredient from another component of the composition.

The term “erodible” as used herein refers to formulations which may be worn away, diminished, or deteriorated by the action of substances within the body.

The term “diffusion controlled” as used herein refers to formulations which may spread as the result of their spontaneous movement, for example, from a region of higher to one of lower concentration.

The term “osmotic controlled” as used herein refers to formulations which may spread as the result of their movement through a semi-permeable membrane into a solution of higher concentration that tends to equalize the concentrations of the formulation on the two sides of the membrane.

### 1. Nanoparticulate Compositions Comprising a Platelet Aggregation Inhibitor

The present invention provides a nanoparticulate composition comprising particles which comprise: (A) a platelet aggregation inhibitor; and (B) at least one surface stabilizer. Examples of platelet aggregation inhibitors for use in the present invention include cilostazol, and salts and derivatives thereof. Nanoparticualte compositions were first described in U.S. Pat. No. 5,145,684. Nanoparticulate active agent compositions are described also in, for example, U.S. Pat. Nos. 5,298,262; 5,302,401; 5,318,767; 5,326,552; 5,328,404; 5,336,507; 5,340,564; 5,346,702; 5,349,957; 5,352,459; 5,399,363; 5,494,683; 5,401,492; 5,429,824; 5,447,710; 5,451,393; 5,466,440; 5,470,583; 5,472,683; 5,500,204; 5,518,738; 5,521,218; 5,525,328; 5,543,133; 5,552,160; 5,565,188; 5,569,448; 5,571,536; 5,573,749; 5,573,750; 5,573,783; 5,580,579; 5,585,108; 5,587,143; 5,591,456; 5,593,657; 5,622,938; 5,628,981; 5,643,552; 5,718,388; 5,718,919; 5,747,001; 5,834,025; 6,045,829; 6,068,858; 6,153,225; 6,165,506; 6,221,400; 6,264,922; 6,267,989; 6,270,806; 6,316,029; 6,375,986; 6,428,814; 6,431,478; 6,432,381; 6,582,285; 6,592,903; 6,656,504; 6,742,734; 6,745,962; 6,811,767; 6,908,626; 6,969,529; 6,976;647; and 6,991,191; and U.S. Patent Publication Nos. 20020012675; 20050276974; 20050238725; 20050233001; 20050147664; 20050063913; 20050042177; 20050031691; 20050019412; 20050004049; 20040258758; 20040258757; 20040229038; 20040208833; 20040195413; 20040156895; 20040156872; 20040141925; 20040115134; 20040105889; 20040105778; 20040101566; 20040057905; 20040033267; 20040033202; 20040018242; 20040015134; 20030232796; 20030215502; 20030185869; 20030181411; 20030137067; 20030108616; 20030095928; 20030087308; 20030023203; 20020179758; and 20010053664. None of the above documents describe nanoparticulate compositions comprising a platelet aggregation inhibitor Amorphous small particle compositions are described, for example, in U.S. Pat. Nos. 4,783,484; 4,826,689; 4,997,454; 5,741,522; 5,776,496.

As stated above, the effective average particle size of the particles in the nanoparticulate composition of the present invention is less than about 2000 m (i.e., 2 microns) in diameter. In embodiments of the present invention, the effective average particle size may be, for example, less than about 1900 nm, less than about 1800 nm, less than about 1700 nm, less than about 1600 nm, less than about 1500 mm, less than about 1400 nm, less than about 1300 nm, less than about 1200 nm, less than about 1100 nm, less than about 1000 mm, less than about 900 mm, less than about 800 nm, less than about 700 mm, less than about 600 mm, less than about 500 nm, less than about 400 mm, less than about 300 nm, less than about 250 nm, less than about 200 mm, less than about 150 mm, less than about 100 nm, less than about 75 mm, or less than about 50 nm in diameter, as measured by light-scattering methods, microscopy, or other appropriate methods.

The nanoparticulate particles may exist in a crystalline phase, an amorphous phase, a semi-crystalline phase, a semi amorphous phase, or a mixture thereof.

In addition to allowing for a smaller solid dosage form size, the nanoparticulate composition of the present invention exhibits increased bioavailability, and requires smaller doses of the platelet aggregation inhibitor as compared to prior conventional, non-nanoparticulate compositions which comprise a platelet aggregation inhibitor. In one embodiment of the invention, the platelet aggregation inhibitor, when administered in the nanoparticulate composition of the present invention, has a bioavailability that is about 50% greater than the platelet aggregation inhibitor when administered in a conventional dosage form. In other embodiments, the platelet aggregation inhibitor, when administered in the nanoparticulate composition of the present invention, has a bioavailability that is about 40% greater, about 30% greater, about 20% or about 10% greater than the platelet aggregation inhibitor when administered in a conventional dosage form.

The nanoparticulate composition preferably also has a desirable pharmacokinetic profile as measured following the initial dosage thereof to a mammalian subject. The desirable pharmacokinetic profile of the composition includes, but is not limited to: (1) a Cₘₐₓ for the platelet aggregation inhibitor, when assayed in the plasma of a mammalian subject following administration, that is preferably greater than the Cₘₐₓ for the same platelet aggregation inhibitor delivered at the same dosage by a non-nanoparticulate composition; and/or (2) an AUC for the platelet aggregation inhibitor, when assayed in the plasma of a mammalian subject following administration, that is preferably greater than the AUC for the same platelet aggregation inhibitor delivered at the same dosage by a non-nanoparticulate composition; and/or (3) a Tₘₐₓ for the platelet aggregation inhibitor, when assayed in the plasma of a mammalian subject following administration, that is preferably less than the Tₘₐₓ for the same platelet aggregation inhibitor delivered at the same dosage by a non-nanoparticulate composition.

In an embodiment of the present invention, a nanoparticulate composition of the present invention exhibits, for example, a Tₘₐₓ for a platelet aggregation inhibitor contained therein which is not greater than about 90% of the Tₘₐₓ for the same platelet aggregation inhibitor delivered at the same dosage by a non-nanoparticulate composition. In other embodiments of the present invention, the nanoparticulate composition of the present invention may exhibit, for example, a Tₘₐₓ for a platelet aggregation inhibitor contained therein which is not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ for the same platelet aggregation inhibitor delivered at the same dosage by a non-nanoparticulate composition.

In an embodiment of the present invention, a nanoparticulate composition of the present invention exhibits, for example, a Cₘₐₓ for a platelet aggregation inhibitor contained therein which is at least about 50% of the Cₘₐₓ for the same platelet aggregation inhibitor delivered at the same dosage by a non-nanoparticulate composition. In other embodiments of the present invention, the nanoparticulate composition of the present invention may exhibit, for example, a Cₘₐₓ for a platelet aggregation inhibitor contained therein which is at least about 100%, at least about 200%, at least about 300%, at least about 400%, at least about 500%, at least about 600%, at least about 700%, at least about 800%, at least about 900%, at least about 1100%, at least about 1100%, at least about 1200%, at least about 1300%, at least about 1400%, at least about 1500%, at least about 1600%, at least about 1700%, at least about 1800%, or at least about 1900% greater than the Cₘₐₓ for the same platelet aggregation inhibitor delivered at the same dosage by a non-nanoparticulate composition.

In an embodiment of the present invention, a nanoparticulate composition of the present invention exhibits, for example, an AUC for a platelet aggregation inhibitor contained therein which is at least about 25% greater than the AUC for the same platelet aggregation inhibitor delivered at the same dosage by a non-nanoparticulate composition. In other embodiments of the present invention, the nanoparticulate composition of the present invention may exhibit, for example, an AUC for a platelet aggregation inhibitor contained therein which is at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC for the same platelet aggregation inhibitor delivered at the same dosage by a non-nanoparticulate composition.

The invention encompasses a nanoparticulate composition wherein the pharmacokinetic profile of the platelet aggregation inhibitor following administration is not substantially affected by the fed or fasted state of a subject ingesting the composition. This means that there is no substantial difference in the quantity of platelet aggregation inhibitor absorbed or the rate of platelet aggregation inhibitor absorption when the nanoparticulate composition is administered in the fed versus the fasted state. In conventional cilostazol formulations, i.e., PLETAL®, the absorption of cilostazol is increased when administered with food. This difference in absorption observed with conventional cilostazol formulations is undesirable. The composition of the invention overcomes this problem.

Benefits of a dosage form which substantially eliminates the effect of food include an increase in subject convenience, thereby increasing subject compliance, as the subject does not need to ensure that they are taking a dose either with or without food. This is significant, as with poor subject compliance an increase in the medical condition for which the platelet aggregation inhibitor is being prescribed may be observed, i.e., ischemic symptoms for poor subject compliance with platelet aggregation inhibitor.

The invention encompasses also a nanoparticulate composition comprising the platelet aggregation inhibitor in which administration of the composition to a subject in a fasted state is bioequivalent to administration of the composition to a subject in a fed state.

The difference in absorption of the composition of the invention, when administered in the fed versus the fasted state, preferably is less than about 60%, less than about 55%, less than about 50%, less than about 45%, less than about 40%, less than about 35%, less than about 30%, less than about 25%, less than about 20%, less than about 15%, less than about 10%, less than about 5%, or less than about 3%.

In one embodiment of the invention, the invention encompasses a composition comprising the platelet aggregation inhibitor wherein the administration of the composition to a subject in a fasted state is bioequivalent to administration of the composition to a subject in a fed state, in particular as defined by Cₘₐₓ and AUC guidelines given by the U.S. Food and Platelet aggregation inhibitor Administration and the corresponding European regulatory agency (EMEA). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% Cl for AUC must be between 0.80 to 1.25 and the 90% Cl for Cₘₐₓ must between 0.70 to 1.43.

The nanoparticulate composition of the invention is proposed to have an unexpectedly dramatic dissolution profile. Rapid dissolution of an administered platelet aggregation inhibitor is preferable, as faster dissolution generally leads to faster onset of action and greater bioavailability. To improve the dissolution profile and bioavailability of the platelet aggregation inhibitor, it would be useful to increase the platelet aggregation inhibitor's dissolution so that it could attain a level close to 100%.

The compositions of the invention preferably have a dissolution profile in which within about 5 minutes at least about 20% of the composition is dissolved. In other embodiments of the invention, at least about 30% or at least about 40% of the composition is dissolved within about 5 minutes. In yet other embodiments of the invention, preferably at least about 40%, at least about 50%, at least about 60%, at least about 70%, or at least about 80% of the composition is dissolved within about 10 minutes. Finally, in another embodiment of the invention, preferably at least about 70%, at least about 80%, at least about 90%, or at least about 100% of the composition is dissolved within about 20 minutes.

Dissolution is preferably measured in a medium which is discriminating. Such a dissolution medium will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

An additional feature of the nanoparticulate composition of the invention is that particles thereof redisperse so that the particles have an effective average particle size of less than about 2000 nm in diameter. This is significant because, if the particles did not redisperse so that they have an effective average particle size of less than about 2000 nm in diameter, the composition may lose the benefits afforded by formulating the platelet aggregation inhibitor therein into a nanoparticulate form. This is because nanoparticulate compositions benefit from the small size of the particles comprising the platelet aggregation inhibitor. If the particles do not redisperse into small particle sizes upon administration, then “clumps” or agglomerated particles are formed, owing to the extremely high surface free energy of the nanoparticulate system and the thermodynamic driving force to achieve an overall reduction in free energy. With the formation of such agglomerated particles, the bioavailability of the dosage form may fall well below that observed with the liquid dispersion form of the nanoparticulate composition.

In other embodiments of the invention, the redispersed particles of the invention (redispersed in water, a biorelevant media, or any other suitable liquid media) have an effective average particle size of less than about less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 mm, less than about 400 nm, less than about 300 nm, less than about 250 nm, less than about 200 nm, less than about 150 nm, less than about 100 mm, less than about 75 mm, or less than about 50 nm in diameter, as measured by light-scattering methods, microscopy, or other appropriate methods. Such methods suitable for measuring effective average particle size are known to a person of ordinary skill in the art.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

The nanoparticulate composition of the present invention exhibits dramatic redispersion of the particles upon administration to a mammal, such as a human or animal, as demonstrated by reconstitution/redispersion in a biorelevant aqueous media, such that the effective average particle size of the redispersed particles is less than about 2000 nm. Such biorelevant aqueous media can be any aqueous media that exhibits the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1 M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997). It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 N, and NaCl solutions, ranging in concentration from about 0.001 to about 0.1 M, and mixtures thereof. For example, electrolyte solutions can be, but are not limited to, about 0.1 N HCl or less, about 0.01 N HCl or less, about 0.001 N HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 N HCl, 0.01 N HCl, and 0.1 N HCl correspond to pH 3, pH 2, and pH 1, respectively. Thus, a 0.01 N HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

As stated above, the composition comprises also at least one surface stabilizer. The surface stabilizer can be adsorbed on or associated with the surface of the platelet aggregation inhibitor. Preferably, the surface stabilizer adheres on, or associates with, the surface of the particles, but does not react chemically with the particles or with other surface stabilizer molecules. Individually adsorbed molecules of the surface stabilizer are essentially free of intermolecular cross-linkages.

The relative amounts of the platelet aggregation inhibitor and surface stabilizer present in the composition of the present invention can vary widely. The optional amount of the individual components can depend, upon, among other things, the particular platelet aggregation inhibitor selected, the hydrophilic-lipophilic balance (HLB), melting point, and the surface tension of water solutions of the stabilizer. The concentration of the platelet aggregation inhibitor can vary from about 99.5% to about 0.001%, from about 95% to about 0.1%, or from about 90% to about 0.5%, by weight, based on the total combined weight of the platelet aggregation inhibitor and surface stabilizer(s), not including other excipients. The concentration of the surface stabilizer(s) can vary from about 0.5% to about 99.999%, from about 5.0% to about 99.9%, or from about 10% to about 99.5%, by weight, based on the total combined dry weight of the platelet aggregation inhibitor and surface stabilizer(s), not including other excipients.

The choice of a surface stabilizer(s) for the platelet aggregation inhibitor is non-trivial and required extensive experimentation to realize a desirable formulation. Accordingly, the present invention is directed to the surprising discovery that nanoparticulate compositions comprising a platelet aggregation inhibitor can be made.

Combinations of more than one surface stabilizer can be used in the invention. Useful surface stabilizers which can be employed in the invention include, but are not limited to, known organic and inorganic pharmaceutical excipients. Such excipienis include various polymers, low molecular weight oligomers, natural products, and surfactants. Surface stabilizers include nonionic, anionic, cationic, ionic, and zwitterionic surfactants.

Representative examples of surface stabilizers include hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-2004, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-IOG® or Surfactant 10-G® (Olin Chemicals, Stamford, Conn.); Crodestas SL-400 (Croda, Inc.); and SA9OHCO, which is C₁₈H₃₇CH₂(CON(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylanumonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quaternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅-dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl(ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyldimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide; choline esters (such as choline esters of fatty acids), benzalkonium chloride, stearalkonium chloride compounds (such as stearyltrimonium chloride and Distearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quaternary phosphorous compound, a pyridinium compound, an anilinium compound, an ammonium compound, a hydroxylammonium compound, a primary ammonium compound, a secondary ammonium compound, a tertiary ammonium compound, and quaternary ammonium compounds of the formula NR₁R₂R₃R₁⁽⁺⁾. For compounds of the formula NR₁R₂R₃R₄⁽⁺⁾:
(i) none of R₁-R₄ are CH₃;
(ii) one of R₁-R₄ is CH₃;
(iii) three of R₁-R₄ are CH₃;
(iv) all of R₁-R₄ are CH₃;
(v) two of R₁-R₄ are CH₃, one of R₁-R₄ is C₆H₅CH₂, and one of R₁-R₄ is an alkyl chain of seven carbon atoms or less;
(vi) two of R₁-R₄ are CH₃, one of R₁-R₄ is C₆H₅CH₂, and one of R₁-R₄ is an alkyl chain of nineteen carbon atoms or more;
(vii) two of R₁-R₄ are CH₃ and one of R₁-R₄ is the group C₆H₅(CH₂)ₙ, where n>1;
(viii) two of R₁-R₄ are CH₃, one of R₁-R₄ is C₆H₅CH₂, and one of R₁-R₄ comprises at least one heteroatom;
(ix) two of R₁-R₄ are CH₃, one of R₁-R₄ is C₆H₅CH₂, and one of R₁-R₄ comprises at least one halogen;
(x) two of R₁-R₄ are CH₃, one of R₁-R₄ is C₆H₅CH₂, and one of R₁-R₄ comprises at least one cyclic fragment;
(xi) two of R₁-R₄ are CH₃ and one of R₁-R₄ is a phenyl ring; or
(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface stabilizers are commercially available and/or can be prepared by techniques known in the art. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

The compositions of the invention can comprise, in addition to the platelet aggregation inhibitor, one or more compounds useful in treating ischemic symptoms. The composition may also be administered in conjunction with such a compound. Examples of such compounds include, but are not limited to, prostaglandins and derivatives thereof, thrombolytic agents, anticoagulants, calcium-entry blocking agents, antianginal agents, cardiac glycosides, vasodilators, antihypertensive agents, and blood lipid-lowering agents.

The composition of the present invention may comprise also one or more binding agents, filling agents, diluents, lubricating agents, emulsifying and suspending agents, sweeteners, flavoring agents, preservatives, buffers, wetting agents, disintegrants, effervescent agents, perfuming agents, and other excipients. Such excipients are known in the art. In addition, prevention of the growth of microorganisms can be ensured by the addition of various antibacterial and antifungal agents, such as parabens, chlorobutanol, phenol, sorbic acid, and the like. For use in injectable formulations, the composition may comprise also isotonic agents, such as sugars, sodium chloride, and the like and agents for use in delaying the absorption of the injectable pharmaceutical form, such as aluminum monostearate and gelatin.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The composition of the present invention may comprise also a carrier, adjuvant, or a vehicle (hereafter, collectively, “carriers”).

The nanoparticulate compositions can be made using, for example, milling, homogenization, precipitation, freezing, or template emulsion techniques. Exemplary methods of making nanoparticulate compositions are described in the '684 patent. Methods of making nanoparticulate compositions are described also in U.S. Pat. Nos. 5,518,187; 5,718,388; 5,862,999; 5,665,331; 5,662,883; 5,560,932; 5,543,133; 5,534,270; 5,510,118; and 5,470,583.

In one method, particles comprising the platelet aggregation inhibitor are dispersed in a liquid dispersion medium in which the platelet aggregation inhibitor is poorly soluble. Mechanical means are then used in the presence of grinding media to reduce the particle size to the desired effective average particle size. The dispersion medium can be, for example, water, safflower oil, ethanol, t-butanol, glycerin, polyethylene glycol (PEG), hexane, or glycol. A preferred dispersion medium is water. The particles can be reduced in size in the presence of at least one surface stabilizer. The particles comprising the platelet aggregation inhibitor can be contacted with one or more surface stabilizers after attrition. Other compounds, such as a diluent, can be added to the platelet aggregation inhibitor/surface stabilizer composition during the size reduction process. Dispersions can be manufactured continuously or in a batch mode. One skilled in the art would understand that it may be the case that, following milling, not all particles may be reduced to the desired size. In such an event, the particles of the desired size may be separated and used in the practice of the present invention.

Another method of forming the desired nanoparticulate composition is by microprecipitation. This is a method of preparing stable dispersions of poorly soluble platelet aggregation inhibitor in the presence of surface stabilizer(s) and one or more colloid stability-enhancing surface active agents free of any trace toxic solvents or solubilized heavy metal impurities. Such a method comprises, for example: (1) dissolving the platelet aggregation inhibitor in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

A nanoparticulate composition may be formed also by homogenization. Exemplary homogenization methods are described in U.S. Pat. No. 5,510,118, for “Process of Preparing Therapeutic Compositions Containing Nanoparticles.” Such a method comprises dispersing particles comprising the platelet aggregation inhibitor in a liquid dispersion medium, followed by subjecting the dispersion to homogenization to reduce the particle size to the desired effective average particle size. The particles can be reduced in size in the presence of at least one surface stabilizer. The particles can be contacted with one or more surface stabilizers either before or after attrition. Other compounds, such as a diluent, can be added to the composition before, during, or after the size reduction process. Dispersions can be manufactured continuously or in a batch mode.

Another method of forming the desired nanoparticulate composition is by spray freezing into liquid (SFL). This technology comprises injecting an organic or organoaqueous solution of the platelet aggregation inhibitor and surface stabilizer(s) into a cryogenic liquid, such as liquid nitrogen. The droplets of the platelet aggregation inhibitor-containing solution freeze at a rate sufficient to minimize crystallization and particle growth, thus formulating nano-structured particles. Depending on the choice of solvent system and processing conditions, the particles can have varying particle morphology. In the isolation step, the nitrogen and solvent are removed under conditions that avoid agglomeration or ripening of the particles.

As a complementary technology to SFL, ultra rapid freezing (URF) may also be used to create equivalent nanostructured particles with greatly enhanced surface area. URF comprises taking a water-miscible, anhydrous, organic, or organoaqueous solution of the platelet aggregation inhibitor and surface stabilizer(s) and applying it onto a cryogenic substrate. The solvent is then removed by means such as lyophilization or atmospheric freeze-drying with the resulting nanostructured particles remaining.

Another method of forming the desired nanoparticulate composition is by template emulsion. Template emulsion creates nano-structured particles with controlled particle size distribution and rapid dissolution performance. The method comprises preparing an oil-in-water emulsion and then swelling it with a non-aqueous solution comprising the platelet aggregation inhibitor and surface stabilizer(s). The size distribution of the particles is a direct result of the size of the emulsion droplets prior to loading of the emulsion with the platelet aggregation inhibitor. The particle size can be controlled and optimized in this process. Furthermore, through selected use of solvents and stabilizers, emulsion stability is achieved with no or suppressed Ostwald ripening. Subsequently, the solvent and water are removed, and the stabilized nano-structured particles are recovered. Various particle morphologies can be achieved by appropriate control of processing conditions.

The invention provides a method comprising the administration of an effective amount of a nanoparticulate composition comprising the platelet aggregation inhibitor.

The composition of the present invention can be formulated for administration parentally (e.g., intravenous, intramuscular, or subcutaneous), orally (e.g., in solid, liquid, or aerosol form, vaginal), nasally, rectally, oticly, ocularly, locally (e.g., in powder, ointment, or drop form), buccally, intracistemally, intraperitoneally, or topically, and the like.

The nanoparticulate composition can be utilized in solid or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

Compositions suitable for parenteral injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

Solid dosage forms for oral administration include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. A solid dose tablet formulation is preferred. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the platelet aggregation inhibitor, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

One of ordinary skill will appreciate that a therapeutically effective amount of the platelet aggregation inhibitor can be determined empirically. The platelet aggregation inhibitor may be a compound, for example cilostazol, in pure form or, where such forms exist, in pharmaceutically acceptable salt, ester, or proplatelet aggregation inhibitor form. Actual dosage levels of the platelet aggregation inhibitor in the nanoparticulate compositions of the invention may be varied to obtain an amount of the platelet aggregation inhibitor that is effective to obtain a desired therapeutic response for a particular composition and method of administration. The selected dosage level therefore depends upon the desired therapeutic effect, the route of administration, the potency of the administered platelet aggregation inhibitor, the desired duration of treatment, and other factors.

Dosage unit compositions may contain such amounts of the platelet aggregation inhibitor or such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the platelet aggregation inhibitor; the duration of the treatment; active compound used in combination or coincidental with the platelet aggregation inhibitor; and like factors well known in the medical arts.

### II. Controlled Release Platelet Aggregation Inhibitor Compositions

As used in the present application, the term “active agent” may refer to the platelet aggregation inhibitor, nanoparticles comprising the platelet aggregation inhibitor, or any other compound that has a pharmaceutical affect.

The effectiveness of pharmaceutical compounds in the prevention and treatment of disease states depends on a variety of factors including the rate and duration of delivery of the compound from the dosage form to the patient. The combination of delivery rate and duration exhibited by a given dosage form in a patient can be described as its in vivo release profile and, depending on the pharmaceutical compound administered, will be associated with a concentration and duration of the pharmaceutical compound in the blood plasma, referred to as a plasma profile. As pharmaceutical compounds vary in their pharmacokinetic properties such as bioavailability, and rates of absorption and elimination, the release profile and the resultant plasma profile become important elements to consider in designing effective therapies.

The release profiles of dosage forms may exhibit different rates and durations of release and may be continuous or pulsatile. Continuous release profiles include release profiles in which a quantity of one or more pharmaceutical compounds is released continuously throughout the dosing interval at either a constant or variable rate. Pulsatile release profiles include release profiles in which at least two discrete quantities of one or more pharmaceutical compounds are released at different rates and/or over different time frames. For any given pharmaceutical compound or combination of such compounds, the release profile for a given dosage form gives rise to an associated plasma profile in a patient. When two or more components of a dosage form have different release profiles, the release profile of the dosage form as a whole is a combination of the individual release profiles and may be described generally as “multimodal.” The release profile of a two-component dosage form in which each component has a different release profile may described as “bimodal,” and the release profile of a three-component dosage form in which each component has a different release profile may described as “trimodal.”

Similar to the variables applicable to the release profile, the associated plasma profile in a patient may exhibit constant or variable blood plasma concentration levels of the pharmaceutical compounds over the duration of action and may be continuous or pulsatile. Continuous plasma profiles include plasma profiles of all rates and duration which exhibit a single plasma concentration maximum. Pulsatile plasma profiles include plasma profiles in which at least two higher blood plasma concentration levels of pharmaceutical compound are separated by a lower blood plasma concentration level and may be described generally as “multimodal.” Pulsatile plasma profiles exhibiting two peaks may be described as “bimodal” and plasma profiles exhibiting three peaks may be described as “trimodal.” Depending on, at least in part, the pharmacokinetics of the pharmaceutical compounds included in the dosage form as well as the release profiles of the individual components of the dosage form, a multimodal release profile may result in either a continuous or a pulsatile plasma profile upon administration to a patient.

In one embodiment, the present invention provides a multiparticulate modified release composition which delivers platelet aggregation inhibitor, or nanoparticles containing the platelet aggregation inhibitor, in a pulsatile manner. The nanoparticles are of the type described above and comprise also at least one surface stabilizer.

In still another embodiment, the present invention provides a multiparticulate modified release composition which delivers the platelet aggregation inhibitor, or nanoparticles containing the platelet aggregation inhibitor, in a continuous manner. The nanoparticles are of the type described above and comprise also at least one surface stabilizer.

In yet another embodiment, the present invention provides a multiparticulate modified release composition in which a first portion of the platelet aggregation inhibitor, or nanoparticles containing the platelet aggregation inhibitor, is released immediately upon administration and one or more subsequent portions of the platelet aggregation inhibitor, or nanoparticles containing the platelet aggregation inhibitor, are released after an initial time delay.

In yet another embodiment, the present invention provides solid oral dosage forms for once-daily or twice-daily administration comprising the multiparticulate modified release composition of the present invention.

In still another embodiment, the present invention provides a method for the prevention and/or treatment of ischemic symptoms comprising the administration of a composition of the present invention.

In an embodiment, the present invention provides a multiparticulate modified release composition in which the particles forming the multiparticulate are nanoparticulate particles of the type described above. The nanoparticulate particles may, as desired, contain a modified release coating and/or a modified release matrix material.

In an embodiment, the platelet aggregation inhibitor used in the compositions described herein is cilostazol or its salts or derivatives.

According to one aspect of the present invention, there is provided a pharmaceutical composition having a first component comprising active ingredient-containing particles, and at least one subsequent component comprising active ingredient-containing particles, each subsequent component having a rate and/or duration of release different from the first component wherein at least one of said components comprises particles containing platelet aggregation inhibitor. In an embodiment of the invention, the platelet aggregation inhibitor-containing particles that form the multiparticulate may themselves contain nanoparticulate particles of the type described above which comprise the platelet aggregation inhibitor and also at least one surface stabilizer. In another embodiment of the invention, nanoparticulate particles of the type described above which comprise the platelet aggregation inhibitor and also at least one surface stabilizer themselves are the platelet aggregation inhibitor-containing particles of the multiparticulate. The platelet aggregation inhibitor-containing particles may be coated with a modified release coating. Alternatively or additionally, the platelet aggregation inhibitor-containing particles may comprise a modified release matrix material. Following oral delivery, the composition delivers the platelet aggregation inhibitor, or nanoparticles containing the platelet aggregation inhibitor, in a pulsatile manner. In one embodiment, the first component provides an immediate release of the platelet aggregation inhibitor, or nanoparticles containing the platelet aggregation inhibitor, and the one or more subsequent components provide a modified release of the platelet aggregation inhibitor, or nanoparticles containing the platelet aggregation inhibitor. In such embodiments, the immediate release component serves to hasten the onset of action by minimizing the time from administration to a therapeutically effective plasma concentration level, and the one or more subsequent components serve to minimize the variation in plasma concentration levels and/or maintain a therapeutically effective plasma concentration throughout the dosing interval.

The modified release coating and/or the modified release matrix material cause a lag time between the release of the active ingredient from the first population of active ingredient-containing particles and the release of the active ingredient from subsequent populations of active ingredient-containing particles. Where more than one population of active ingredient-containing particles provide a modified release, the modified release coating and/or the modified release matrix material causes a lag time between the release of the active ingredient from the different populations of active ingredient-containing particles. The duration of these lag times may be varied by altering the composition and/or the amount of the modified release coating and/or altering the composition and/or amount of modified release matrix material utilized. Thus, the duration of the lag time can be designed to mimic a desired plasma profile.

Because the plasma profile produced by the modified release composition upon administration is substantially similar to the plasma profile produced by the administration of two or more IR dosage forms given sequentially, the modified release composition of the present invention is particularly useful for administering a platelet aggregation inhibitor, for example cilostazol or its salts and derivatives.

According to another aspect of the present invention, the composition can be designed to produce a plasma profile that minimizes or eliminates the variations in plasma concentration levels associated with the administration of two or more IR dosage forms given sequentially. In such embodiments, the composition may be provided with an immediate release component to hasten the onset of action by minimizing the time from administration to a therapeutically effective plasma concentration level, and at least one modified release component to maintain a therapeutically effective plasma concentration level throughout the dosing interval.

The active ingredients in each component may be the same or different. For example, the composition may comprise components comprising only the platelet aggregation inhibitor, or nanoparticles containing the platelet aggregation inhibitor, as the active ingredient. Alternatively, the composition may comprise a first component comprising the platelet aggregation inhibitor, or nanoparticles containing the platelet aggregation inhibitor, and at least one subsequent component comprising an active ingredient other than the platelet aggregation inhibitor, or nanoparticles containing the platelet aggregation inhibitor, suitable for co-administration with the platelet aggregation inhibitor, or a first component containing an active ingredient other than the platelet aggregation inhibitor, or nanoparticles containing the platelet aggregation inhibitor, and at least one subsequent component comprising the platelet aggregation inhibitor, or nanoparticles containing the platelet aggregation inhibitor. Indeed, two or more active ingredients may be incorporated into the same component when the active ingredients are compatible with each other. An active ingredient present in one component of the composition may be accompanied by, for example, an enhancer compound or a sensitizer compound in another component of the composition, in order to modify the bioavailability or therapeutic effect thereof.

As used herein, the term “enhancer” refers to a compound which is capable of enhancing the absorption and/or bioavailability of an active ingredient by promoting net transport across the GIT in an animal, such as a human. Enhancers include but are not limited to medium chain fatty acids; salts, esters, ethers and derivatives thereof, including glycerides and triglycerides; non-ionic surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures of two or more of these agents.

In those embodiments in which more than one platelet aggregation inhibitor-containing component is present, the proportion of platelet aggregation inhibitor contained in each component may be the same or different depending on the desired dosing regime. The platelet aggregation inhibitor present in the first component and in subsequent components may be any amount sufficient to produce a therapeutically effective plasma concentration level. The platelet aggregation inhibitor, when applicable, may be present either in the form of one substantially optically pure stereoisomer or as a mixture, racemic or otherwise, of two or more stereoisomers. The platelet aggregation inhibitor is preferably present in the composition in an amount of from about 0.1 to about 500 mg, preferably in the amount of from about 1 to about 100 mg. The platelet aggregation inhibitor is preferably present in the first component in an amount of from about 0.5 to about 60 mg; more preferably the platelet aggregation inhibitor, is present in the first component in an amount of from about 2.5 to about 30 mg. The platelet aggregation inhibitor is present in subsequent components in an amount within similar ranges to those described for the first component.

The time release characteristics for the delivery of the platelet aggregation inhibitor from each of the components may be varied by modifying the composition of each component, including modifying any of the excipients and/or coatings which may be present. In particular, the release of the platelet aggregation inhibitor, or nanoparticles containing the platelet aggregation inhibitor, may be controlled by changing the composition and/or the amount of the modified release coating on the particles, if such a coating is present. If more than one modified release component is present, the modified release coating for each of these components may be the same or different. Similarly, when modified release is facilitated by the inclusion of a modified release matrix material, release of the active ingredient may be controlled by the choice and amount of modified release matrix material utilized. The modified release coating may be present, in each component, in any amount that is sufficient to yield the desired delay time for each particular component. The modified release coating may be preset, in each component, in any amount that is sufficient to yield the desired time lag between components.

The lag time and/or time delay for the release of the platelet aggregation inhibitor from each component may also be varied by modifying the composition of each of the components, including modifying any excipients and coatings which may be present. For example, the first component may be an immediate release component wherein the platelet aggregation inhibitor, or nanoparticles containing the platelet aggregation inhibitor, is released immediately upon administration. Alternatively, the first component may be, for example, a time-delayed immediate release component in which the platelet aggregation inhibitor, or nanoparticles containing the platelet aggregation inhibitor, is released substantially in its entirety immediately after a time delay. The second and subsequent component may be, for example, a time-delayed immediate release component as just described or, alternatively, a time-delayed sustained release or extended release component in which the platelet aggregation inhibitor, or nanoparticles containing the platelet aggregation inhibitor, is released in a controlled fashion over an extended period of time.

As will be appreciated by those skilled in the art, the exact nature of the plasma concentration curve will be influenced by the combination of all of these factors just described. In particular, the lag time between the delivery (and thus also the onset of action) of the platelet aggregation inhibitor, or nanoparticles containing the platelet aggregation inhibitor, in each component may be controlled by varying the composition and coating (if present) of each of the components. Thus by variation of the composition of each component (including the amount and nature of the active ingredient(s)) and by variation of the lag time, numerous release and plasma profiles may be obtained. Depending on the duration of the lag time between the release of the platelet aggregation inhibitor, or nanoparticles containing the platelet aggregation inhibitor, from each component and the nature of the release of the platelet aggregation inhibitor, or nanoparticles containing the platelet aggregation inhibitor, from each component (i.e. immediate release, sustained release etc.), the plasma profile may be continuous (i.e., having a single maximum) or pulsatile in which the peaks in the plasma profile may be well separated and clearly defined (e.g. when the lag time is long) or superimposed to a degree (e.g. when the lag time is short).

The plasma profile produced from the administration of a single dosage unit comprising the composition of the present invention is advantageous when it is desirable to deliver two or more pulses of active ingredient without the need for administration of two or more dosage units.

Any coating material which modifies the release of the platelet aggregation inhibitor in the desired manner may be used. In particular, coating materials suitable for use in the practice of the present invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the trademark Eudragit® RS and RL, poly acrylic acid and poly acrylate and methacrylate copolymers such as those sold under the trademark Eudragit® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, polyvinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate) (mol. wt. ˜5 k-5,000 k), polyvinylpyrrolidone (mol. wt. ˜10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (mol. wt. ˜30 k-300 k), polysaccharides such as agar, acacia, karaya, tragacanth, algins and guar, polyacrylamides, Polyox® polyethylene oxides (mol. wt. ˜100 k-5,000 k), AquaKeep® acrylate polymers, diesters of polyglucan, crosslinked polyvinyl alcohol and poly N-vinyl-2-pyrrolidone, sodium starch glucolate (e.g. Explotab®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticisers, lubricants, solvents and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

When the modified release component comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of a platelet aggregation inhibitor dispersed therein in vitro or in vivo. Modified release matrix materials suitable for the practice of the present invention include but are not limited to microcrystalline cellulose, sodium carboxymethylcellulose, hydroxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate and mixture thereof.

A modified release composition according to the present invention may be incorporated into any suitable dosage form which facilitates release of the active ingredient in a pulsatile manner. In one embodiment, the dosage form comprises a blend of different populations of active ingredient-containing particles which make up the immediate release and the modified release components, the blend being filled into suitable capsules, such as hard or soft gelatin capsules. Alternatively, the different individual populations of active ingredient-containing particles may be compressed (optionally with additional excipients) into mini-tablets which may be subsequently filled into capsules in the appropriate proportions. Another suitable dosage form is that of a multilayer tablet. In this instance the first component of the modified release composition may be compressed into one layer, with the second component being subsequently added as a second layer of the multilayer tablet. The populations of the particles making up the composition of the invention may further be included in rapidly dissolving dosage forms such as an effervescent dosage form or a fast-melt dosage form.

In one embodiment, the composition comprises at least two platelet aggregation inhibitor-containing components: a first platelet aggregation inhibitor-containing component and one or more subsequent platelet aggregation inhibitor-containing components. In such embodiment, the first platelet aggregation inhibitor-containing component of the composition may exhibit a variety of release profiles including profiles in which substantially all of the platelet aggregation inhibitor, or nanoparticles containing the platelet aggregation inhibitor, contained in the first component is released rapidly upon administration of the dosage form, released rapidly but after a time delay (delayed release), or released slowly over time. In one such embodiment, the platelet aggregation inhibitor, or nanoparticles containing the platelet aggregation inhibitor, contained in the first component is released rapidly upon administration to a patient. As used herein, “released rapidly” includes release profiles in which at least about 80% of the active ingredient of a component is released within about an hour after administration, the term “delayed release” includes release profiles in which the active ingredient of a component is released (rapidly or slowly) after a time delay, and the terms “controlled release” and “extended release” include release profiles in which at least about 80% of the active ingredient contained in a component is released slowly.

The second platelet aggregation inhibitor-containing component of such embodiment may also exhibit a variety of release profiles including an immediate release profile, a delayed release profile or a controlled release profile. In one such embodiment, the second platelet aggregation inhibitor-containing component exhibits a delayed release profile in which the platelet aggregation inhibitor of the component, or nanoparticles containing the platelet aggregation inhibitor, is released after a time delay.

The plasma profile produced by the administration of dosage forms of the present invention which comprise an immediate release component comprising the platelet aggregation inhibitor, or nanoparticles containing the platelet aggregation inhibitor, and at least one modified release component comprising the platelet aggregation inhibitor, or nanoparticles containing the platelet aggregation inhibitor, can be substantially similar to the plasma profile produced by the administration of two or more IR dosage forms given sequentially, or to the plasma profile produced by the administration of separate IR and modified release dosage forms. Accordingly, the dosage forms of the present invention can be particularly useful for administering platelet aggregation inhibitor where the maintenance of pharmacokinetic parameters may be desired but is problematic.

In one embodiment, the composition and the solid oral dosage forms containing the composition release the platelet aggregation inhibitor, or nanoparticles containing the platelet aggregation inhibitor, such that substantially all of the platelet aggregation inhibitor, or nanoparticles containing the platelet aggregation inhibitor, contained in the first component is released prior to release of the platelet aggregation inhibitor, or nanoparticles containing the platelet aggregation inhibitor, from the at least one subsequent component. When the first component comprises an IR component, for example, it is preferable that release of the platelet aggregation inhibitor, or nanoparticles containing the platelet aggregation inhibitor, from the at least one second component is delayed until substantially all the platelet aggregation inhibitor, or nanoparticles containing the platelet aggregation inhibitor, in the IR component has been released. Release of the platelet aggregation inhibitor, or nanoparticles containing the platelet aggregation inhibitor, from the at least one subsequent component may be delayed as detailed above by the use of a modified release coatings and/or a modified release matrix material.

When it is desirable to minimize patient tolerance by providing a dosage regime which facilitates wash-out of a first dose of the platelet aggregation inhibitor from a patient's system, release of the platelet aggregation inhibitor, or nanoparticles containing the platelet aggregation inhibitor, from subsequent components may be delayed until substantially all of the platelet aggregation inhibitor, or nanoparticles containing the platelet aggregation inhibitor, contained in the first component has been released, and further delayed until at least a portion the platelet aggregation inhibitor released from the first component has been cleared from the patient's system. In one embodiment, release of the platelet aggregation inhibitor, or nanoparticles containing the platelet aggregation inhibitor, from subsequent components of the composition is substantially, if not completely, delayed for a period of at least about two hours after administration of the composition. In another embodiment, the release of platelet aggregation inhibitor, or nanoparticles containing the platelet aggregation inhibitor, from subsequent components of the composition is substantially, if not completely, delayed for a period of at least about four hours after administration of the composition.

As described hereinbelow, the present invention also includes various types of modified release systems by which the platelet aggregation inhibitor, or nanoparticles containing the platelet aggregation inhibitor, may be delivered in either a pulsatile or continuous manner. These systems include but are not limited to: films with the platelet aggregation inhibitor, or nanoparticles containing the platelet aggregation inhibitor, in a polymer matrix (monolithic devices); systems in which the platelet aggregation inhibitor, or nanoparticles containing the platelet aggregation inhibitor, is contained by a polymer (reservoir devices); polymeric colloidal particles or microencapsulates (microparticles, microspheres or nanoparticles) in the form of reservoir and matrix devices; systems in which the platelet aggregation inhibitor, or nanoparticles containing the platelet aggregation inhibitor, is contained by a polymer which contains a hydrophilic and/or leachable additive e.g., a second polymer, surfactant or plasticizer, etc. to give a porous device, or a device in which platelet aggregation inhibitor release may be osmotically controlled (both reservoir and matrix devices); enteric coatings (ionizable and dissolve at a suitable pH); (soluble) polymers with (covalently) attached pendant platelet aggregation inhibitor molecules; and devices where release rate is controlled dynamically: e.g., the osmotic pump.

The delivery mechanism of the present invention can control the rate of release of platelet aggregation inhibitor, or nanoparticles containing the platelet aggregation inhibitor. While some mechanisms will release platelet aggregation inhibitor, or nanoparticles containing the platelet aggregation inhibitor, at a constant rate, others will vary as a function of time depending on factors such as changing concentration gradients or additive leaching leading to porosity, etc.

Polymers used in sustained release coatings are necessarily biocompatible, and ideally biodegradable. Examples of both naturally occurring polymers such as Aquacoat® (FMC Corporation, Food & Pharmaceutical Products Division, Philadelphia, USA) (ethylcellulose mechanically spheronised to sub-micron sized, aqueous based, pseudo-latex dispersions), and also synthetic polymers such as the Eudragit® (Röhm Pharma, Weiterstadt.) range of poly(acrylate, methacrylate) copolymers are known in the art.

Reservoir Devices

A typical approach to modified release is to encapsulate or contain the platelet aggregation inhibitor, or nanoparticles containing the platelet aggregation inhibitor, entirely (e.g., as a core), within a polymer film or coat (i.e., microcapsules or spray/pan coated cores).

The various factors that can affect the diffusion process may readily be applied to reservoir devices (e.g., the effects of additives, polymer functionality (and, hence, sink-solution pH) porosity, film casting conditions, etc.) and, hence, the choice of polymer must be an important consideration in the development of reservoir devices. Modeling the release characteristics of reservoir devices (and monolithic devices) in which the transport of the platelet aggregation inhibitor is by a solution-diffusion mechanism therefore typically involves a solution to Fick's second law (unsteady-state conditions; concentration dependent flux) for the relevant boundary conditions. When the device contains dissolved active agent, the rate of release decreases exponentially with time as the concentration (activity) of the agent (i.e., the driving force for release) within the device decreases (i.e., first order release). If, however, the active agent is in a saturated suspension, then the driving force for release is kept constant until the device is no longer saturated. Alternatively the release-rate kinetics may be desorption controlled, and a function of the square root of time.

Transport properties of coated tablets, may be enhanced compared to free-polymer films, due to the enclosed nature of the tablet core (permeant) which may enable the internal build-up of an osmotic pressure which will then act to force the permeant out of the tablet.

The effect of de-ionized water on salt containing tablets coated in poly(ethylene glycol) (PEG)-containing silicone elastomer, and also the effects of water on free films has been investigated. The release of salt from the tablets was found to be a mixture of diffusion through water filled pores, formed by hydration of the coating, and osmotic pumping. KCl transport through films containing just 10% PEG was negligible, despite extensive swelling observed in similar free films, indicating that porosity was necessary for the release of the KCl which then occurred by trans-pore diffusion. Coated salt tablets, shaped as disks, were found to swell in de-ionized water and change shape to an oblate spheroid as a result of the build-up of internal hydrostatic pressure: the change in shape providing a means to measure the force generated. As might be expected, the osmotic force decreased with increasing levels of PEG content. The lower PEG levels allowed water to be imbibed through the hydrated polymer, while the porosity resulting from the coating dissolving at higher levels of PEG content (20 to 40%) allow the pressure to be relieved by the flow of KCl.

Methods and equations have been developed, which by monitoring (independently) the release of two different salts (e.g., KCl and NaCl) allowed the calculation of the relative magnitudes that both osmotic pumping and trans-pore diffusion contributed to the release of salt from the tablet. At low PEG levels, osmotic flow was increased to a greater extent than was trans-pore diffusion due to the generation of only a low pore number density: at a loading of 20%, both mechanisms contributed approximately equally to the release. The build-up of hydrostatic pressure, however, decreased the osmotic inflow, and osmotic pumping. At higher loadings of PEG, the hydrated film was more porous and less resistant to outflow of salt. Hence, although the osmotic pumping increased (compared to the lower loading), trans-pore diffusion was the dominant release mechanism. An osmotic release mechanism has also been reported for microcapsules containing a water soluble core.

Monolithic Devices (Matrix Devices)

Monolithic (matrix) devices may be used for controlling the release of platelet aggregation inhibitors, or nanoparticles containing the platelet aggregation inhibitor. This is possibly because they are relatively easy to fabricate compared to reservoir devices, and the danger of an accidental high dosage that could result from the rupture of the membrane of a reservoir device is not present. In such a device, the active agent is present as a dispersion within the polymer matrix, and they are typically formed by the compression of a polymer/platelet aggregation inhibitor mixture or by dissolution or melting. The dosage release properties of monolithic devices may be dependent upon the solubility of the platelet aggregation inhibitor, or nanoparticles containing the platelet aggregation inhibitor, in the polymer matrix or, in the case of porous matrixes, the solubility in the sink solution within the particle's pore network, and also the tortuosity of the network (to a greater extent than the permeability of the film), dependent on whether the platelet aggregation inhibitor, or nanoparticles containing the platelet aggregation inhibitor, is dispersed in the polymer or dissolved in the polymer. For low loadings of platelet aggregation inhibitor, or nanoparticles containing the platelet aggregation inhibitor, (0 to 5% W/V), the platelet aggregation inhibitor, or nanoparticles containing the platelet aggregation inhibitor, will be released by a solution-diffusion mechanism (in the absence of pores). At higher loadings (5 to 10% W/V), the release mechanism will be complicated by the presence of cavities formed near the surface of the device as the platelet aggregation inhibitor, or nanoparticles containing the platelet aggregation inhibitor, is lost: such cavities fill with fluid from the environment increasing the rate of release of the platelet aggregation inhibitor.

It is common to add a plasticizer (e.g., a poly(ethylene glycol)), a surfactant, or adjuvant (i.e., an ingredient which increases effectiveness), to matrix devices (and reservoir devices) as a means to enhance the permeability (although, in contrast, plasticizers may be fugitive, and simply serve to aid film formation and, hence, decrease permeability—a property normally more desirable in polymer paint coatings). It was noted that the leaching of PEG increased the permeability of (ethyl cellulose) films linearly as a function of PEG loading by increasing the porosity, however, the films retained their barrier properties, not permitting the transport of electrolyte. It was deduced that the enhancement of their permeability was as a result of the effective decrease in thickness caused by the PEG leaching. This was evidenced from plots of the cumulative permeant flux per unit area as a function of time and film reciprocal thickness at a PEG loading of 50% W/W: plots showing a linear relationship between the rate of permeation and reciprocal film thickness, as expected for a (Fickian) solution-diffusion type transport mechanism in a homogeneous membrane. Extrapolation of the linear regions of the graphs to the time axis gave positive intercepts on the time axis: the magnitude of which decreased towards zero with decreasing film thickness. These changing lag times were attributed to the occurrence of two diffusional flows during the early stages of the experiment (the flow of the platelet aggregation inhibitor and also the flow of the PEG), and also to the more usual lag time during which the concentration of permeant in the film is building-up. Caffeine, when used as a permeant, showed negative lag times. No explanation of this was forthcoming, but it was noted that caffeine exhibited a low partition coefficient in the system, and that this was also a feature of aniline permeation through polyethylene films which showed a similar negative time lag.

The effects of added surfactants on (hydrophobic) matrix devices has been investigated. It was thought that surfactant may increase the release rate of a platelet aggregation inhibitor, or nanoparticles containing the platelet aggregation inhibitor, by three possible mechanisms: (i) increased solubilization, (ii) improved ‘wettability’ to the dissolution media, and (iii) pore formation as a result of surfactant leaching. For the system studied (Eudragit® RL 100 and RS 100 plasticised by sorbitol, flurbiprofen as the platelet aggregation inhibitor, and a range of surfactants) it was concluded that improved wetting of the tablet led to only a partial improvement in platelet aggregation inhibitor release (implying that the release was diffusion, rather than dissolution, controlled), although the effect was greater for Eudragit® RS than Eudragit® RL, while the greatest influence on release was by those surfactants that were more soluble due to the formation of disruptions in the matrix allowing the dissolution medium access to within the matrix. This is of obvious relevance to a study of latex films which might be suitable for pharmaceutical coatings, due to the ease with which a polymer latex may be prepared with surfactant as opposed to surfactant-free. Differences were found between the two polymers with only the Eudragit® RS showing interactions between the anionic/cationic surfactant and platelet aggregation inhibitor. This was ascribed to the differing levels of quaternary ammonium ions on the polymer.

Composite devices consisting of a polymer/platelet aggregation inhibitor matrix coated in a polymer containing no platelet aggregation inhibitor also exist. Such a device was constructed from aqueous Eudragit® lattices, and was found to provide a continuous release by diffusion of the platelet aggregation inhibitor from the core through the shell. Similarly, a polymer core containing the platelet aggregation inhibitor has been produced and coated with a shell that was eroded by gastric fluid. The rate of release of the platelet aggregation inhibitor was found to be relatively linear (a function of the rate limiting diffusion process through the shell) and inversely proportional to the shell thickness, whereas the release from the core alone was found to decrease with time.

Microspheres

Methods for the preparation of hollow microspheres have been described. Hollow microspheres were formed by preparing a solution of ethanol/dichloromethane containing the platelet aggregation inhibitor and polymer. On pouring into water, an emulsion is formed containing the dispersed polymer/platelet aggregation inhibitor/solvent particles, by a coacervation-type process from which the ethanol rapidly diffused precipitating polymer at the surface of the droplet to give a hard-shelled particle enclosing the platelet aggregation inhibitor dissolved in the dichloromethane. A gas phase of dichloromethane was then generated within the particle which, after diffusing through the shell, was observed to bubble to the surface of the aqueous phase. The hollow sphere, at reduced pressure, then filled with water which could be removed by a period of drying. No platelet aggregation inhibitor was found in the water. Highly porous matrix-type microspheres have also been described. The matrix-type microspheres were prepared by dissolving the platelet aggregation inhibitor and polymer in ethanol. On addition to water, the ethanol diffused from the emulsion droplets to leave a highly porous particle. A suggested use of the microspheres was as floating platelet aggregation inhibitor delivery devices for use in the stomach.

Pendent Devices

A means of attaching a range of drugs such as analgesics and antidepressants, etc., by means of an ester linkage to poly(acrylate) ester latex particles prepared by aqueous emulsion polymerization has been developed. These lattices, when passed through an ion exchange resin such that the polymer end groups were converted to their strong acid form, could self-catalyze the release of the platelet aggregation inhibitor by hydrolysis of the ester link.

Drugs have been attached to polymers, and also monomers have been synthesized with a pendent platelet aggregation inhibitor attached. Dosage forms have been prepared in which the platelet aggregation inhibitor is bound to a biocompatible polymer by a labile chemical bond e.g., polyanhydrides prepared from a substituted anhydride (itself prepared by reacting an acid chloride with the drug: methacryloyl chloride and the sodium salt of methoxy benzoic acid) were used to form a matrix with a second polymer (Eudragit® RL) which released the drug on hydrolysis in gastric fluid. The use of polymeric Schiff bases suitable for use as carriers of pharmaceutical amines has also been described.

Enteric Films

Enteric coatings consist of pH sensitive polymers. Typically the polymers are carboxylated and interact very little with water at low pH, while at high pH the polymers ionize causing swelling or dissolving of the polymer. Coatings can therefore be designed to remain intact in the acidic environment of the stomach, protecting either the platelet aggregation inhibitor from this environment or the stomach from the platelet aggregation inhibitor, but to dissolve in the more alkaline environment of the intestine.

Osmotically Controlled Devices

The osmotic pump is similar to a reservoir device but contains an osmotic agent (e.g., the active agent in salt form) which acts to imbibe water from the surrounding medium via a semi-permeable membrane. Such a device, called an elementary osmotic pump, has been described. Pressure is generated within the device which forces the active agent out of the device via an orifice of a size designed to minimize solute diffusion, while preventing the build-up of a hydrostatic pressure head which can have the effect of decreasing the osmotic pressure and changing the dimensions of the device. While the internal volume of the device remains constant, and there is an excess of solid or saturated solution in the device, then the release rate remains constant delivering a volume equal to the volume of solvent uptake.

Electrically Stimulated Release Devices

Monolithic devices have been prepared using polyelectrolyte gels which swell when, for example, an external electrical stimulus is applied causing a change in pH. The release may be modulated by changes in the applied current to produce a constant or pulsatile release profile.

Hydrogels

In addition to their use in platelet aggregation inhibitor matrices, hydrogels find use in a number of biomedical applications such as, for example, soft contact lenses, and various soft implants, and the like.

### Methods of Using Modified Release Platelet Aggregation Inhibitor Compositions

According to another aspect of the present invention, there is provided a method for treating a patient suffering from pain and/or inflammation comprising the step of administering a therapeutically effective amount of the platelet aggregation inhibitor composition of the present invention in solid oral dosage form. Advantages of the method of the present invention include a reduction in the dosing frequency required by conventional multiple IR dosage regimes while still maintaining the benefits derived from a pulsatile plasma profile or eliminating or minimizing the variations in plasma concentration levels. This reduced dosing frequency is advantageous in terms of patient compliance and the reduction in dosage frequency made possible by the method of the present invention would contribute to controlling health care costs by reducing the amount of time spent by health care workers on the administration of platelet aggregation inhibitors.

In the following examples, all percentages are weight by weight unless otherwise stated. The term “purified water” as used throughout the Examples refers to water that has been purified by passing it through a water filtration system. It is to be understood that the examples are for illustrative purposes only, and should not be interpreted as restricting the spirit and breadth of the invention as defined by the scope of the claims that follow.

### EXAMPLES

Examples 1 to 3 provide exemplary cilostazol tablet formulations. These examples are not intended to limit the claims in any respect, but rather to provide exemplary tablet formulations of cilostazol which can be utilized in the methods of the invention. Such exemplary tablets can also comprise a coating agent.

### Example 1

Exemplary Nanoparticulate Cilostazol Tablet Formulation #1

| Component | g/Kg |
|---|---|
| Cilostazol | about 50 to about 500 |
| Hypromellose, USP | about 10 to about 70 |
| Docusate Sodium, USP | about 1 to about 10 |
| Sucrose, NF | about 100 to about 500 |
| Sodium Lauryl Sulfate, NF | about 1 to about 40 |
| Lactose Monohydrate, NF | about 50 to about 400 |
| Silicified Microcrystalline Cellulose | about 50 to about 300 |
| Crospovidone, NF | about 20 to about 300 |
| Magnesium Stearate, NF | about 0.5 to about 5 |

### Example 2

Exemplary Nanoparticulate Cilostazol Tablet Formulation #2

| Component | g/Kg |
|---|---|
| Cilostazol | about 100 to about 300 |
| Hypromellose, USP | about 30 to about 50 |
| Docusate Sodium, USP | about 0.5 to about 10 |
| Sucrose, NF | about 100 to about 300 |
| Sodium Lauryl Sulfate, NF | about 1 to about 30 |
| Lactose Monohydrate, NF | about 100 to about 300 |
| Silicified Microcrystalline Cellulose | about 50 to about 200 |
| Crospovidone, NF | about 50 to about 200 |
| Magnesium Stearate, NF | about 0.5 to about 5 |

### Example 3

Exemplary Nanoparticulate Cilostazol Tablet Formulation #3

| Component | g/Kg |
|---|---|
| Cilostazol | about 200 to about 225 |
| Hypromellose, USP | about 42 to about 46 |
| Docusate Sodium, USP | about 2 to about 6 |
| Sucrose, NF | about 200 to about 225 |
| Sodium Lauryl Sulfate, NF | about 12 to about 18 |
| Lactose Monohydrate, NF | about 200 to about 205 |
| Silicified Microcrystalline Cellulose | about 130 to about 135 |
| Crospovidone, NF | about 112 to about 118 |
| Magnesium Stearate, NF | about 0.5 to about 3 |

### Example 4

### Multiparticulate Modified Release Composition Containing Cilostazol

A multiparticulate modified release composition according to the present invention comprising an immediate release component and a modified release component containing cilostazol is prepared as follows.

(a) Immediate Release Component.

A solution of cilostazol (50:50 racemic mixture) is prepared according to any of the formulations given in Table 1. The methylphenidate solution is then coated onto nonpareil seeds to a level of approximately 16.9% solids weight gain using, for example, a Glatt GPCG3 (Glatt, Protech Ltd., Leicester, UK) fluid bed coating apparatus to form the IR particles of the immediate release component.

TABLE 1
Immediate release component solutions

| | Amount, | % (w/w) |
|---|---|---|
| Ingredient | (i) | (ii) |
| Cilostazol | 13.0 | 13.0 |
| Polyethylene Glycol 6000 | 0.5 | 0.5 |
| Polyvinylpyrrolidone | 3.5 | |
| Purified Water | 83.5 | 86.5 |

(b) Modified Release Component

Cilostazol-containing delayed release particles are prepared by coating immediate release particles prepared according to Example 1(a) above with a modified release coating solution as detailed in Table 2. The immediate release particles are coated to varying levels up to approximately to 30% weight gain using, for example, a fluid bed apparatus.

TABLE 2
Modified release component coating solutions

| | Amount, % (w/w) | | | | | | | |
|---|---|---|---|---|---|---|---|---|
| Ingredient | (i) | (ii) | (iii) | (iv) | (v) | (vi) | (vii) | (viii) |
| Eudragit ® | 49.7 | 42.0 | 47.1 | 53.2 | 40.6 | — | — | 25.0 |
| RS 12.5 | | | | | | | | |
| Eudragit ® | — | — | — | — | — | 54.35 | 46.5 | — |
| S 12.5 | | | | | | | | |
| Eudragit ® | — | — | — | — | — | — | 25.0 | |
| L 12.5 | | | | | | | | |
| Polyvinyl- | — | — | — | 0.35 | 0.3 | — | — | |
| pyrrolidone | | | | | | | | |
| Diethyl- | 0.5 | 0.5 | 0.6 | 1.35 | 0.6 | 1.3 | 1.1 | — |
| phthalate | | | | | | | | |
| Triethyl- | — | — | — | — | — | — | — | 1.25 |
| citrate | | | | | | | | |
| Isopropyl | 39.8 | 33.1 | 37.2 | 45.1 | 33.8 | 44.35 | 49.6 | 46.5 |
| alcohol | | | | | | | | |
| Acetone | 10.0 | 8.3 | 9.3 | — | 8.4 | — | — | — |
| Talc¹ | — | 16.0 | 5.9 | — | 16.3 | — | 2.8 | 2.25 |

¹Talc is simultaneously applied during coating for formulations in column (i), (iv) and (vi).

(c) Encapsulation of Immediate and Delayed Release Particles.

The immediate and delayed release particles prepared according to Example 1(a) and (b) above are encapsulated in size 2 hard gelatin capsules to an overall 20 mg dosage strength using, for example, a Bosch GKF 4000S encapsulation apparatus. The overall dosage strength of 20 mg cilostazol was made up of 10 mg from the immediate release component and 10 mg from the modified release component.

### Example 5

### Multiparticulate Modified Release Composition Containing Cilostazol

Multiparticulate modified release cilostazol compositions according to the present invention having an immediate release component and a modified release component having a modified release matrix material are prepared according to the formulations shown in Table 3(a) and (b).

TABLE 3 (a)
100 mg of IR component is encapsulated with 100 mg of modified
release (MR) component to give a 20 mg dosage strength product

% (w/w)

IR component

| | |
|---|---|
| Cilostazol | 10 |
| Microcrytalline cellulose | 40 |
| Lactose | 45 |
| Povidone | 5 |

MR component

| | |
|---|---|
| Cilostazol | 10 |
| Microcrytalline cellulose | 40 |
| Eudragit ® RS | 45 |
| Povidone | 5 |

TABLE 3 (b)
50 mg of IR component is encapsulated with 50 mg of modified release
(MR) component to give a 20 mg dosage strength product.

% (w/w)

IR component

| | |
|---|---|
| Cilostazol | 20 |
| Microcrystalline cellulose | 50 |
| Lactose | 28 |
| Povidone | 2 |

MR component

| | |
|---|---|
| Cilostazol | 20 |
| Microcrytalline cellulose | 50 |
| Eudragit ® S | 28 |
| Povidone | 2 |

### Example 6

The purpose of this example was to prepare nanoparticulate cilostazol compositions using various combinations of surface stabilizers and milling times.

An aqueous dispersion of cilostazol combined with one or more surface stabilizers, at the concentrations shown in Table 4, below, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). All compositions were milled for 60 min. at a mill speed of 2500 rpm.

TABLE 4
Cilostazol Formulations

| | | | Deionized |
|---|---|---|---|
| | Cilostazol | | Water |
| Sample | Concentration | Surface Stabilizer(s) | (w/w) |
| 1 | 5% (w/w) | Pharmacoat 603, 1.25% (w/w) (Hydroxypropyl | 93.75 |
| | | methylcellulose) | |
| 2 | 5% (w/w) | HPC-SL, 2% (w/w) (Hydroxypropyl Cellulose - Super | 93 |
| | | Low Viscosity) | |
| 3 | 5% (w/w) | HPC-SL, 1.25% (w/w) (Hydroxypropyl Cellulose - Super | 93.7 |
| | | Low Viscosity) | |
| | | Docusate Sodium, 0.05% (w/w) (Docusate Sodium) | |
| 4 | 5% (w/w) | Plasdone K-17, 1.25% (w/w) (Povidone K-17) | 93.7 |
| | | Benzalkonium Chloride, 0.05% (w/w) (Benzalkonium | |
| | | Chloride) | |
| 5 | 5% (w/w) | Tween 80, 1% (w/w) (Polyoxyethylene Sorbitan Fatty | 94 |
| | | Acid Ester) | |
| 6 | 5% (w/w) | Tween 80, 1.5% (w/w) (Polyoxyethylene Sorbitan Fatty | 93.45 |
| | | Acid Ester) | |
| | | Lecithin, 0.05% (w/w) | |
| 7 | 5% (w/w) | Lutrol F68, 1.25% (w/w) (Poloxamer 188) | 93.7 |
| | | Docusate Sodium, 0.05% (w/w) (Docusate Sodium) | |
| 8 | 5% (w/w) | Plasdone C-15, 1.25% (w/w) (Povidone C-15) | 93.7 |
| | | Deoxycholate acid, Sodium salt, 0.05% (w/w) | |
| 9 | 5% (w/w) | Tyloxapol, 1% (w/w) | 94.0 |
| 10 | 5% (w/w) | Plasdone S-630, 1.25% (w/w) (Povidone | 93.7 |
| | | Sodium Lauryl Sulfate, 0.05% (w/w) (Sodium Lauryl | |
| | | Sulfate) | |
| 11 | 5% (w/w) | Lutrol F127, 1.5% (w/w) (Poloxamer) | 93.5 |
| 12 | 5% (w/w) | Pharmacoat 603, 1.25% (w/w) (Hydroxypropyl | 93.7 |
| | | Methylcellulose) | |
| | | Docusate Sodium, 0.05% (w/w) (Docusate Sodium) | |
| 13 | 5% (w/w) | Plasdone S-630, 1.25% (w/w) (Povidone) | 93.75 |
| 14 | 5% (w/w) | Pluronic F108 (poloxamer) 1.25% (w/w) | 92.5 |
| | | Tween 80, 1.25% (w/w) (Polyoxyethylene Sorbitan Fatty | |
| | | Acid Ester) | |
| 15 | 5% (w/w) | Plasdone K29/32, 1.25% (w/w) | 93.7 |
| | | Sodium Lauryl Sulfate, 0.05% (w/w) | |
| 16 | 5% (w/w) | Plasdone S-630, 2% (w/w) | 93 |
| 17 | 5% (w/w) | Pharmacoat 603 2% (w/w) | 93 |
| 18 | 5% (w/w) | Docusate sodium, 0.1% (w/w) | 94.9 |
| 19 | 5% (w/w) | Pluronic F108, 1.5% (w/w) | 93.5 |
| 20 | 5% (w/w) | Sodium lauryl sulfate, 0.1% (w/w) | 94.9 |
| 21 | 5% (w/w) | Plasdone K29/32, 2% (w/w) | 93 |

The milled compositions were analyzed via microscopy. Microscopy was done using a Lecia DM5000B and Lecia CTR 5000 light source (Laboratory Instruments and Supplies Ltd., Ashbourne Co., Meath, Ireland). The microscopy observations for each formulation are shown below in Table 5.

TABLE 5

| Formulation | Microscopy Observations |
|---|---|
| 1 | The sample appeared in places to be well dispersed with discrete nanoparticles |
| | of cilostazol present with Brownian motion evident. However, flocculation of |
| | cilostazol particles was clearly present throughout the sample. |
| 2 | The sample appeared well dispersed with nanoparticles of cilostazol present. |
| | Brownian motion was also clearly evident. There was no sign of cilostazol |
| | particle flocculation or crystal growth throughout the slide. |
| 3 | The sample appeared well dispersed with discrete nanoparticles of cilostazol |
| | clearly visible. Brownian motion was also clearly evident with no signs of |
| | cilostazol particle flocculation or crystal growth. |
| 4 | Microscopy showed the sample to be well dispersed with nanoparticles of |
| | cilostazol clearly visible. Brownian motion was also observed. There may |
| | have been some signs of partially milled platelet aggregation inhibitor. There |
| | was no sign of flocculation. |
| 5 | Microscopy showed that the sample is composed of nanoparticles of cilostazol |
| | which exhibited Brownian motion. Cilostazol particle flocculation was not |
| | apparent when analyzing the sample under the microscope. Large cilostazol |
| | particles were observed throughout the sample. These were identified to |
| | approximately 1 micron in size. |
| 6 | Microscopy showed the sample to be well dispersed with nanoparticles of |
| | cilostazol clearly visible. Brownian motion was also clearly observed. There |
| | may have been some signs of partially milled platelet aggregation inhibitor but |
| | no signs of cilostazol particle flocculation. |
| 7 | This sample appeared well dispersed with nanoparticles of cilostazol present. |
| | Brownian motion was also clearly evident. Some larger platelet aggregation |
| | inhibitor particles were observed through out the sample, but these were no |
| | bigger than 1000 nm in size. There were no signs of cilostazol crystal growth |
| | or cilostazol particle flocculation. |
| 8 | There were some nanoparticles of cilostazol present in this sample. There were |
| | also some evidence of Brownian motion. However, most of the sample showed |
| | severe cilostazol particle flocculation. |
| 9 | This sample appeared very well dispersed with nanoparticles of cilostazol |
| | visible. Brownian motion was also clearly evident. There were no signs of un- |
| | milled platelet aggregation inhibitor particles, cilostazol particle flocculation or |
| | crystal growth |
| 10 | This sample appeared very well dispersed with nanoparticles of cilostazol |
| | visible. Brownian motion was also clearly evident. There were some un-milled |
| | platelet aggregation inhibitor crystals observed. There were no signs of crystal |
| | growth or cilostazol particle flocculation. |
| 11 | Microscopy showed the presence of discrete nanoparticles of cilostazol all of |
| | which appeared to exhibit Brownian motion. There was no apparent cilostazol |
| | particle flocculation observed when analyzing the diluted sample slurry under |
| | the x 100 oil phase objective. A small proportion of the sample showed some |
| | unmilled platelet aggregation inhibitor particles but this appeared to be in small |
| | amounts. |
| 12 | Microscopy showed the presence of discrete nanoparticles of cilostazol which |
| | exhibited Brownian motion. There was no flocculation observed during analysis |
| | of the samples. The aliquot of sample slurry analysed appeared to be well |
| | dispersed |
| 13 | Nanoparticles of cilostazol were observed in this sample. Brownian motion was |
| | also evident. However, a majority of the sample showed severe cilostazol |
| | particle flocculation. There were no signs of un-milled platelet aggregation |
| | inhibitor or cilostazol crystal growth. |
| 14 | This sample appeared well dispersed with nanoparticles of cilostazol present. |
| | Brownian motion was also clearly evident. There were no signs of cilostazol |
| | crystal growth or cilostazol particle flocculation. |
| 15 | Microscopy showed the sample to be well dispersed composed of nanoparticles |
| | of cilostazol. Brownian motion was also clearly evident. There was no |
| | evidence of cilostazol particle flocculation. There was no sign of cilostazol |
| | crystal growth. |
| 16 | Microscopy showed the sample to be highly flocculated, as it was evident in the |
| | particle size analysis. Nanoparticles of cilostazol were also clearly visible. |
| | Brownian motion also evident. |
| 17 | The sample clearly showed signs of flocculation as flocculates could be seen |
| | across the whole sample. In such flocculated zones, no Brownian motion could |
| | be observed, while some was seen in non-flocculated zones. Microscopy |
| | observation supports particle size analysis results: there are signs of flocculation |
| | occurring in this formulation. |
| 18 | Microscopy showed the sample had flocculation in high amounts as well as |
| | Brownian motion which was also observed. The particle size analysis showed |
| | that this flocculation could be drastically reduced by sonication. |
| 19 | Microscopy showed the sample had nanoparticles of cilostazol clearly visible. |
| | Brownian motion was also clearly observed. There may have been signs of |
| | partially milled platelet aggregation inhibitor particles but no signs of cilostazol |
| | particle flocculation. Particle size analysis for D50 shows that particle size of |
| | less than 2000 nm was achieved for the D50. |
| 20 | The sample displayed discrete cilostazol nanoparticles which were well |
| | dispersed. Brownian motion was clearly evident. There was no evidence of |
| | any cilostazol particle flocculation or cilostazol crystal growth present. The |
| | particle size analysis pre/post sonification showed high level of flocculation, |
| | which is not supporting the microscopy observation. This flocculation seen in |
| | the particle size analysis may be caused by a higher dilution than with the |
| | microscopy analysis. |
| 21 | It is apparent from microscopy that this sample is indeed largely flocculated. |
| | There are some localized areas where nanoparticles of cilostazol are observed |
| | which exhibit Brownian motion but there are in very minute proportions. |
| | Overall this sample is largely flocculated with particles that appear stagnant. |

The particle size of the milled cilostazol particles was measured, in Milli Q Water, using a Horiba-LA-910 Particle Sizer (Particular Sciences, Hatton Derbyshire, England). Cilostazol particle size was measured initially and then again following 60 seconds sonication. The results are shown below in Table 6.

TABLE 6

| | Mean | D50 | D90 | D95 | | |
|---|---|---|---|---|---|---|
| Sample | (nm) | (nm) | (nm) | (nm) | Sonication? | Comments |
| 1 | 2027 | 298 | 8730 | 13370 | N | Particle size analysis and |
| | 213 | 201 | 281 | 323 | Y | microscopy were performed |
| | | | | | | on harvested material after |
| | | | | | | the 60 min milling |
| | | | | | | processing. |
| 2 | 208 | 200 | 273 | 303 | N | Particle size analysis and |
| | 207 | 199 | 272 | 301 | Y | microscopy were performed |
| | | | | | | on harvested material after |
| | | | | | | the 60 min milling |
| | | | | | | processing. |
| 3 | 224 | 214 | 291 | 329 | N | Particle size analysis and |
| | 223 | 213 | 290 | 327 | Y | microscopy were performed |
| | | | | | | on harvested material after |
| | | | | | | the 60 min milling |
| | | | | | | processing. |
| 4 | 287 | 279 | 385 | 428 | N | Particle size analysis and |
| | 199 | 181 | 293 | 351 | Y | microscopy were performed |
| | | | | | | on harvested material after |
| | | | | | | the 60 min milling |
| | | | | | | processing. |
| 5 | 214 | 206 | 278 | 309 | N | Particle size analysis and |
| | 215 | 207 | 279 | 310 | Y | microscopy were performed |
| | | | | | | on harvested material after |
| | | | | | | the 60 min milling |
| | | | | | | processing. |
| 6 | 194 | 188 | 249 | 275 | N | Particle size analysis and |
| | 195 | 189 | 251 | 277 | Y | microscopy were performed |
| | | | | | | on harvested material after |
| | | | | | | the 60 min milling |
| | | | | | | processing. |
| 8 | 5524 | 4685 | 10609 | 13456 | N | Particle size analysis and |
| | 433 | 312 | 793 | 1267 | Y | microscopy were performed |
| | | | | | | on harvested material after |
| | | | | | | the 60 min milling |
| | | | | | | processing. |
| 9 | 204 | 196 | 263 | 293 | N | Particle size analysis and |
| | 205 | 197 | 265 | 294 | Y | microscopy were performed |
| | | | | | | on harvested material after |
| | | | | | | the 60 min milling |
| | | | | | | processing. |
| 10 | 227 | 217 | 294 | 329 | N | Particle size analysis and |
| | 228 | 218 | 295 | 331 | Y | microscopy were performed |
| | | | | | | on harvested material after |
| | | | | | | the 60 min milling |
| | | | | | | processing. |
| 11 | 363 | 326 | 556 | 618 | N | The particle size analysis |
| | 353 | 325 | 525 | 1860 | Y | was repeated (on another |
| | | | | | | day as day of experiment)/ |
| | | | | | | Particle size analysis and |
| | | | | | | microscopy were performed |
| | | | | | | on harvested material after |
| | | | | | | the 60 min milling |
| | | | | | | processing. |
| | 678 | 427 | 1482 | 1662 | N | Repeat of particle size |
| | 620 | 410 | 1338 | 310 | Y | analysis because of lamp |
| | | | | | | transmittance issue, without |
| | | | | | | any additional processing. |
| | | | | | | The increase in particle size |
| | | | | | | data (D50 and other |
| | | | | | | indicators) may be |
| | | | | | | explained by crystal growth |
| | | | | | | between the two analysis |
| | | | | | | (performed on different |
| | | | | | | days). |
| 12 | 189 | 176 | 266 | 310 | N | Particle size analysis and |
| | 190 | 178 | 268 | 312 | Y | microscopy were performed |
| | | | | | | on harvested material after |
| | | | | | | the 60 min milling |
| | | | | | | processing. |
| 13 | 23345 | 14744 | 51772 | 73834 | N | Particle size analysis and |
| | 200 | 178 | 298 | 376 | Y | microscopy were performed |
| | | | | | | on harvested material after |
| | | | | | | the 60 min milling |
| | | | | | | processing. |
| 14 | 306 | 296 | 424 | 472 | N | Particle size analysis and |
| | 307 | 297 | 425 | 473 | Y | microscopy were performed |
| | | | | | | on harvested material after |
| | | | | | | the 60 min milling |
| | | | | | | processing. |
| 15 | 184 | 173 | 254 | 291 | N | Particle size analysis and |
| | 185 | 174 | 256 | 293 | Y | microscopy were performed |
| | | | | | | on harvested material after |
| | | | | | | the 60 min milling |
| | | | | | | processing. |
| 16 | 15819 | 12790 | 29505 | 37869 | N | The particle size analysis |
| | 290 | 280 | 397 | 442 | Y | was thus repeated (on same |
| | 19222 | 12530 | 28926 | 37750 | N | day)/Particle size analysis |
| | 326 | 306 | 466 | 540 | Y | and microscopy were |
| | | | | | | performed on harvested |
| | | | | | | material after the 60 min |
| | | | | | | milling processing. |
| 17 | 333 | 304 | 477 | 572 | N | Particle size analysis and |
| | 195 | 175 | 288 | 360 | Y | microscopy were performed |
| | | | | | | on harvested material after |
| | | | | | | the 60 min milling |
| | | | | | | processing. |
| 18 | 5669 | 5348 | 12809 | 15058 | N | Particle size analysis and |
| | 544 | 283 | 1468 | 2146 | Y | microscopy were performed |
| | | | | | | on harvested material after |
| | | | | | | the 60 min milling |
| | | | | | | processing. |
| 19 | 328 | 306 | 474 | 551 | N | Particle size analysis and |
| | 320 | 304 | 451 | 512 | Y | microscopy were performed |
| | | | | | | on harvested material after |
| | | | | | | the 60 min milling |
| | | | | | | processing. |
| 20 | 7941 | 7789 | 17310 | 20398 | N | Particle size analysis and |
| | 652 | 294 | 1859 | 2648 | Y | microscopy were performed |
| | | | | | | on harvested material after |
| | | | | | | the 60 min milling |
| | | | | | | processing. |
| 21 | 4601 | 2493 | 12546 | 17197 | N | Particle size analysis and |
| | 346 | 326 | 496 | 570 | Y | microscopy were performed |
| | | | | | | on harvested material after |
| | | | | | | the 60 min milling |
| | | | | | | processing. |

Particle sizes that vary significantly following sonication, such as that observed for Samples 1, 8, 13, 16, 18, 20, and 21 in Table 6, are undesirable, as it is indicative of the presence of cilostazol aggregates. Such aggregates result in compositions having highly variable particle sizes. Such highly variable particle sizes can result in variable absorption between dosages of a platelet aggregation inhibitor, and therefore are undesirable.

The data demonstrate the successful preparation of nanoparticulate cilostazol formulations utilizing various surface stabilizers, including various combination of surface stabilizers.

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

What is claimed is:

1. A stable nanoparticulate composition comprising: (A) particles comprising a platelet aggregation inhibitor, said particles having an effective average particle size of less than about 2000 nm in diameter; and (B) at least one surface stabilizer.

2. The composition of claim 1, wherein said particles are in a crystalline phase, an amorphous phase, a semi-crystalline phase, a semi amorphous phase, or a mixture thereof.

3. The composition of claim 1, wherein the effective average particle size of said particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm in diameter.

4. The composition of claim 1, wherein the composition is formulated:
(A) for administration selected from the group consisting of via injection, oral, vaginal, nasal, rectal, otically, ocular, local, buccal, intracisternal, intraperitoneal, or topically;
(B) into a dosage form selected from the group consisting of tablets, capsules, sachets, solutions, dispersions gels, aerosols, ointments, creams, and mixtures thereof;
(C) into a dosage form selected from the group consisting of controlled release formulations, fast melt formulations, lyophilized formulations, delayed release formulations, extended release formulations, pulsatile release formulations, and mixed immediate release and controlled release formulations; or
(D) any combination of (A), (B), or (C).

5. The composition of claim 1 further comprising one or more pharmaceutically acceptable excipients, carriers, or a combination thereof.

6. The composition of claim 1, wherein:
(A) said platelet aggregation inhibitor is present in said composition in an amount selected from the group consisting of from about 99.5% to about 0.001%, from about 95% to about 0.1%, or from about 90% to about 0.5%, by weight of the total combined dry weight of platelet aggregation inhibitor and surface stabilizer in the composition, not including other excipients;
(B) said surface stabilizer or surface stabilizers are present in a total amount of from about 0.5% to about 99.999%, from about 5.0% to about 99.9%, or from about 10% to about 99.5% by weight, based on the total combined dry weight of platelet aggregation inhibitor and surface stabilizer in the composition not including other excipients; or
(C) a combination of (A) and (B).

7. The composition of claim 1, wherein the surface stabilizer is selected from the group consisting of a non-ionic surface stabilizer, an anionic surface stabilizer, a cationic surface stabilizer, a zwitterionic surface stabilizer, and an ionic surface stabilizer.

8. The composition of claim 1, wherein the surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan: fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, lysozyme, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipid, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅-dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl(ethenoxy)₄ ammonium chloride, lauryl dimethyl(ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUA™, alkyl pyridinium salts; amines, amine salts, amine oxides, imide azolinium salts, protonated quaternary acrylamides, methylated quaternary polymers, and cationic guar.

9. The composition of claim 1, wherein the composition does not produce significantly different absorption levels when administered under fed as compared to fasting conditions.

10. The composition of claim 1, wherein administration of the composition to a subject in a fasted state is bioequivalent to administration of the composition to a subject in a fed state.

11. The composition of claim 1, wherein the pharmacokinetic profile of the composition is not significantly affected by the fed or fasted state of a subject ingesting said composition.

12. A composition according to claim 1 wherein, upon administration of said composition to a mammal, the composition produces therapeutic results at a dosage which is less than that of a non-nanoparticulate dosage form of the same platelet aggregation inhibitor.

13. A composition according to claim 1 which has:
(a) a Cₘₐₓ for the platelet aggregation inhibitor, when assayed in the plasma of a mammalian subject following administration, that is greater than the Cₘₐₓ for the same platelet aggregation inhibitor administered at the same dose using a non-nanoparticulate formulation;
(b) an AUC for the platelet aggregation inhibitor, when assayed in the plasma of a mammalian subject following administration, that is greater than the AUC for the same platelet aggregation inhibitor administered at the same dose using a non-nanoparticulate formulation;
(c) a Tₘₐₓ for the platelet aggregation inhibitor, when assayed in the plasma of a mammalian subject following administration, that is less than the Tₘₐₓ for the same platelet aggregation inhibitor administered at the same dose using a non-nanoparticulate formulation; or
(d) any combination of (a), (b), and (c).

14. The composition of claim 1, additionally comprising one or more active agents useful for the prevention and treatment of ischemic symptoms.

15. The composition of claim 14, wherein the one or more active agents is selected from the group consisting of prostaglandins and derivatives thereof, thrombolytic agents, anticoagulants, calcium-entry blocking agents, anti-anginal agents, cardiac glycosides, vasodilators, antihypertensive agents, and blood lipid-lowering agents.

16. The composition of claim 1 wherein said platelet aggregation inhibitor is cilostazol or a salt or derivative thereof.

17. A method of preparing a nanoparticulate composition comprising a platelet aggregation inhibitor comprising contacting particles comprising said platelet aggregation inhibitor with at least one surface stabilizer for a period of time and under conditions sufficient to provide a nanoparticulate composition comprising a platelet aggregation inhibitor having an effective average particle size of less than about 2000 nm in diameter.

18. The method of claim 17, wherein the contacting comprises grinding, wet grinding, homogenization, precipitation, template emulsion, or supercritical fluid particle generation techniques.

19. The method of claim 17, wherein the effective average particle size of the nanoparticulate particles is selected from the group consisting of less than about 1900 nm, less than about 1800 nm, less than about 1700 nm, less than about 1600 nm, less than about 1500 nm, less than about 1000 nm, less than about 1400 nm, less than about 1300 nm, less than about 1200 nm, less than about 1100 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm in diameter.

20. The method of claim 17 wherein said platelet aggregation inhibitor is cilostazol or a salt or derivative thereof.

21. A method of preventing and/or treating ischemic symptoms comprising administering a composition according to claim 1.

22. The method of claim 21, wherein the effective average particle size of the particles is selected from the group consisting of less than about 1900 nm, less than about 1800 nm, less than about 1700 nm, less than about 1600 nm, less than about 1500 nm, less than about 1000 nm, less than about 1400 nm, less than about 1300 nm, less than about 1200 nm, less than about 1100 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm in diameter.

23. The method of claim 21 wherein said platelet aggregation inhibitor is cilostazol or a salt or derivative thereof.

24. A controlled release composition comprising a population of platelet aggregation inhibitor containing particles, wherein said particles further comprise a modified release coating or, alternatively or additionally, a modified release matrix material, such that the composition following oral delivery to a subject delivers the platelet aggregation inhibitor active in a pulsatile or continuous manner

25. The controlled release composition of claim 24 wherein said platelet aggregation inhibitor is cilostazol or a salt or derivative thereof.

26. The composition according to claim 24, wherein the population comprises modified-release particles.

27. The composition according to claim 24, wherein the population is an erodible formulation.

28. The composition according to claim 24, wherein the modified release particles have a modified-release coating.

29. The composition according to claim 24, wherein the modified release particles comprise a modified-release matrix material.

30. The compositions of claim 28 or 29 wherein said modified release particles are combined in formulation that releases said platelet aggregation inhibitor by erosion to the surrounding environment.

31. The composition according to claim 24, wherein at least one portion of the dose further comprises an enhancer.

32. The composition according to claim 24, wherein the amount of active ingredient contained therein is from about 0.1 mg to about 1 g.

33. The composition according to claim 24 comprising a blend of the particles contained in a hard gelatin or soft gelatin capsule.

34. The composition according to claim 24, wherein the particles are in the form of mini-tablets and the capsule contains a mixture of the mini-tablets.

35. The composition according to claim 24, in the form of tablet comprising layer of compressed particles comprising a platelet aggregate inhibitor.

36. The composition according to claim 24, wherein said particles are provided in a rapidly dissolving dosage form.

37. The composition according to claim 24, comprising a fast-melt tablet.

38. A method for the prevention and/or treatment of ischemic symptoms comprising administering a therapeutically effective amount of a composition according to claim 24.

39. The composition according to claim 24, wherein the modified-release particles comprise a pH-dependent polymer coating which is effective in releasing a pulse of the active ingredient following a time delay of six to twelve hours.

40. The composition according to claim 39, wherein the polymer coating comprises methacrylate copolymers.

41. The composition according to claim 39, wherein the polymer coating comprises a mixture of methacrylate and ammonio methacrylate copolymers in a ratio sufficient to achieve a pulse of release of the active ingredient following a time delay.

42. The composition according to claim 41, wherein the ratio of methacrylate to ammonio methacrylate copolymers is approximately 1:1.

43. A controlled release composition comprising a population of nanoparticulate particles, wherein the nanoparticulate platelet aggregation inhibitor-containing particles comprise a modified release coating or, alternatively or additionally, a modified release matrix material, such that the composition following oral delivery to a subject delivers the platelet aggregation inhibitor in a pulsatile or continuous manner.

44. The composition of claim 43, wherein said composition does not produce significantly different absorption levels when administered under fed as compared to fasting conditions.

45. The composition of claim 43, wherein the pharmacokinetic profile of said composition is not significantly affected by the fed or fasted state of a subject ingesting said composition.

46. The composition of claim 43, wherein administration of said composition to a subject in a fasted state is bioequivalent to administration of said composition to a subject in a fed state.

47. The composition according to claim 43, wherein the population comprises modified-release particles.

48. The composition according to claim 43, wherein the population is an erodible formulation.

49. The composition according to claim 47, wherein the modified release particles have a modified-release coating.

50. The composition according to claim 47, wherein the modified release particles comprise a modified-release matrix material.

51. The compositions of claim 47 wherein said modified release particles are combined in a formulation that releases said platelet aggregation inhibitor by erosion to the surrounding environment.

52. The composition according to claim 43, wherein at least one portion of the dose further comprises an enhancer.

53. The composition according to claim 43, wherein the amount of active ingredient contained therein is from about 0.1 mg to about 1 g.

54. The composition according to claim 43 comprising a blend of the particles contained in a hard gelatin or soft gelatin capsule.

55. The composition according to claim 47, wherein the particles are in the form of mini-tablets and the capsule contains a mixture of the mini-tablets.

56. The composition according to claim 43 in the form of tablet comprising layer of compressed particles which comprise a platelet aggregation inhibitor.

57. The composition according to claim 47, wherein the particles are provided in a rapidly dissolving dosage form.

58. The composition according to claim 43, comprising a fast-melt tablet.

59. The composition according to claim 43 wherein said platelet aggregation inhibitor is cilostazol or a salt or derivative thereof.

59. A method for the prevention and/or treatment of ischemic symptoms comprising administering a therapeutically effective amount of a composition according to claim 43.

60. The composition according to claim 47, wherein the modified-release particles comprise a pH-dependent polymer coating which is effective in releasing a pulse release of the active ingredient following a time delay of six to twelve hours.

61. The composition according to claim 60, wherein the polymer coating comprises methacrylate copolymers.

62. The composition according to claim 60, wherein the polymer coating comprises a mixture of methacrylate and ammonio methacrylate copolymers in a ratio sufficient to achieve a pulse release of the active ingredient following a time delay.

63. The composition according to claim 62, wherein the ratio of methacrylate to ammonio methacrylate copolymers is approximately 1:1.
